SEQUENCE LISTING (22 sequences)

SEQ ID NO: 1 - DNA, 3822 bp, Betacoronavirus, severe acute respiratory syndrome-related coronavirus
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - DNA, 2043 bp, Betacoronavirus, severe acute respiratory syndrome-related coronavirus
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcct

SEQ ID NO: 3 - DNA, 576 bp, Betacoronavirus, severe acute respiratory syndrome-related coronavirus
cctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcacca

SEQ ID NO: 4 - protein, 1276 aa, Betacoronavirus, severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 684 aa, Betacoronavirus, severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 6 - protein, 192 aa, Betacoronavirus, severe acute respiratory syndrome-related coronavirus
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 7 - protein, 69 aa, Saccharomyces cerevisiae
QELTTICEQIPSPTLESTPYSLSTTTILANGKAMQGVFEYYKSVTFVSNCGSHPSTTSKGSPINTQYVF

SEQ ID NO: 8 - protein, 725 aa, Saccharomyces cerevisiae
MTLSFAHFTYLFTILLGLTNIALASDPETILVTITKTNDANGVVTTTVSPALVSTSTIVQAGTTTLYTTWCPLTVSTSSAAEISPSISYATTLSRFSTLTLSTEVCSHEACPSSSTLPTTTLSVTSKFTSYICPTCHTTAISSLSEVGTTTVVSSSAIEPSSASIISPVTSTLSSTTSSNPTTTSLSSTSTSPSSTSTSPSSTSTSSSSTSTSSSSTSTSSSSTSTSPSSTSTSSSLTSTSSSSTSTSQSSTSTSSSSTSTSPSSTSTSSSSTSTSPSSKSTSASSTSTSSYSTSTSPSLTSSSPTLASTSPSSTSISSTFTDSTSSLGSSIASSSTSVSLYSPSTPVYSVPSTSSNVATPSMTSSTVETTVSSQSSSEYITKSSISTTIPSFSMSTYFTTVSGVTTMYTTWCPYSSESETSTLTSMHETVTTDATVCTHESCMPSQTTSLITSSIKMSTKNVATSVSTSTVESSYACSTCAETSHSYSSVQTASSSSVTQQTTSTKSWVSSMTTSDEDFNKHATGKYHVTSSGTSTISTSVSEATSTSSIDSESQEQSSHLLSTSVLSSSSLSATLSSDSTILLFSSVSSLSVEQSPVTTLQISSTSEILQPTSSTAIATISASTSSLSATSISTPSTSVESTIESSSLTPTVSSIFLSSSSAPSSLQTSVTTTEVSTTSISIQYQTSSMVTISQYMGSGSQTRLPLGKLVFAIMAVACNVIFS

SEQ ID NO: 9 - protein, 14 aa, Artificial Sequence
  V5 epitope tag
GKPIPNPLLGLDST

SEQ ID NO: 10 - protein, 18 aa, Saccharomyces cerevisiae
MQLLTCFSIFSVIASVLA

SEQ ID NO: 11 - protein, 15 aa, Artificial Sequence
  (G4S)3 linker
GGGGSGGGGSGGGGS

SEQ ID NO: 12 - protein, 100 aa, Artificial Sequence
  Right flank of construct shown in Figure 1
EFGKPIPNPLLGLDSTGGGGSGGGGSGGGGSQELTTICEQIPSPTLESTPYSLSTTTILANGKAMQGVFEYYKSVTFVSNCGSHPSTTSKGSPINTQYVF

SEQ ID NO: 13 - protein, 1394 aa, Artificial Sequence
  Construct with full S protein as the SARS-CoV-2 antigen
MQLLTCFSIFSVIASVLAMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTEFGKPIPNPLLGLDSTGGGGSGGGGSGGGGSQELTTICEQIPSPTLESTPYSLSTTTILANGKAMQGVFEYYKSVTFVSNCGSHPSTTSKGSPINTQYVF

SEQ ID NO: 14 - protein, 802 aa, Artificial Sequence
  Construct with full S1 subunit as the SARS-CoV-2 antigen
MQLLTCFSIFSVIASVLAMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPEFGKPIPNPLLGLDSTGGGGSGGGGSGGGGSQELTTICEQIPSPTLESTPYSLSTTTILANGKAMQGVFEYYKSVTFVSNCGSHPSTTSKGSPINTQYVF

SEQ ID NO: 15 - protein, 310 aa, Artificial Sequence
  Construct with full RBD subunit as the SARS-CoV-2 antigen
MQLLTCFSIFSVIASVLAPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPEFGKPIPNPLLGLDSTGGGGSGGGGSGGGGSQELTTICEQIPSPTLESTPYSLSTTTILANGKAMQGVFEYYKSVTFVSNCGSHPSTTSKGSPINTQYVF

SEQ ID NO: 16 - DNA, 54 bp, Saccharomyces cerevisiae
atgcagttacttcgctgtttttcaatattttctgttattgcttcagttttagca

SEQ ID NO: 17 - DNA, 42 bp, Artificial Sequence
  Nucleotide sequence for V5 epitope
ggtaagcctatccctaaccctctcctcggtctcgattctacg

SEQ ID NO: 18 - DNA, 45 bp, Artificial Sequence
  Nucleotide sequence for (G4S)3 linker
ggtggtggtggttctggtggtggtggttctggtggtggtggttct

SEQ ID NO: 19 - DNA, 114 bp, Saccharomyces cerevisiae
caggaactgacaactatatgcgagcaaatcccctcaccaactttagaatcgacgccgtactctttgtcaacgactactattttggccaacgggaaggcaatgcaaggagttttt

SEQ ID NO: 20 - DNA, 4077 bp, Artificial Sequence
  Construct using the full S protein as the SARS-CoV-2 antigen
atgcagttacttcgctgtttttcaatattttctgttattgcttcagttttagcaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaggtaagcctatccctaaccctctcctcggtctcgattctacgggtggtggtggttctggtggtggtggttctggtggtggtggttctcaggaactgacaactatatgcgagcaaatcccctcaccaactttagaatcgacgccgtactctttgtcaacgactactattttggccaacgggaaggcaatgcaaggagttttt

SEQ ID NO: 21 - DNA, 2298 bp, Artificial Sequence
  Construct using full S1 subunit as the SARS-CoV-2 antigen
atgcagttacttcgctgtttttcaatattttctgttattgcttcagttttagcaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggtaagcctatccctaaccctctcctcggtctcgattctacgggtggtggtggttctggtggtggtggttctggtggtggtggttctcaggaactgacaactatatgcgagcaaatcccctcaccaactttagaatcgacgccgtactctttgtcaacgactactattttggccaacgggaaggcaatgcaaggagttttt

SEQ ID NO: 22 - DNA, 830 bp, Artificial Sequence
  Construct using full RBD as the SARS-CoV-2 antigen
atgcagttacttcgctgtttttcaatattttctgttattgcttcagttttagcactaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccaggtaagcctatccctaaccctctcctcggtctcgattctacgggtggtggtggttctggtggtggtggttctggtggtggtggttctcaggaactgacaactatatgcgagcaaatcccctcaccaactttagaatcgacgccgtactctttgtcaacgactactattttggccaacgggaaggcaatgcaaggagttttt

(19) United States
(12) Patent Application Publication
(10) Pub. No.: US 2021/0322540 A1
(43) Pub. Date: Oct. 21, 2021

(54) ORAL VACCINES EXPRESSED IN YEAST FOR COVID-19

(71) Applicant: Grapefruit Group Limited, Hong Kong (CH)

(72) Inventors: Dominic Man-Kit Lam, Hong Kong (CN); Jie Liu, Chengdu (CN)

(21) Appl. No.: 16/937,109

(22) Filed: Jul. 23, 2020

Related U.S. Application Data
(60) Provisional application No. 63/030,707, filed on May 27, 2020.
(60) Provisional application No. 63/010,957, filed on Apr. 16, 2020.

(51) Int. Cl. A61K 39/215

(52) U.S. Cl. CPC A61K 39/215; A61K 2039/542

(57) ABSTRACT

Nucleic acid constructs for heterologous expression of a SARS-CoV-2 antigen on the surface of a yeast cell and related polypeptides, recombinant yeast cells, vaccine compositions, oral dosage formulations, and methods of inducing antigen-specific immune response to SARS-CoV-2.

### CROSS-REFERENCE TO RELATED APPLICATIONS

The present application claims priority to U.S. Provisional Patent Application Nos. 63/010,957 (filed Apr. 16, 2020), and 63/030,707 (filed May 27, 2020), the entire contents of each of which are hereby incorporated by reference for all purposes.

### SEQUENCE LISTING

The present specification makes reference to a Sequence Listing (submitted electronically as a .txt file named “MSQ_002_Sequence_Listing.txt” on Aug. 5, 2020). The .txt file was generated on Aug. 4, 2020 and is 68 kb in size. The entire contents of the Sequence Listing are herein incorporated by reference.

### FIELD OF THE INVENTION

The present invention is for the composition of an oral vaccine based on yeast surface display expressions for creating an oral vaccine that prevents and treats infections in animals and humans, including, but not limited to, preventing humans from being infected with Severe Acute Respiratory Syndrome Coronavirus-2 (SARS CoV-2) that causes COVID-19. The present invention comprises mainly an N-terminal yeast surface expression system and oral vaccination in human.

### BACKGROUND OF THE INVENTION

SARS CoV-2 is an emerging infectious pathogen to humans. It is the seventh coronavirus identified and can cause severe respiratory infection. Currently, there are no effective vaccines or adequate treatments available against SARS CoV-2. More importantly, conventional immunization routes such as injection are inconvenient for mass inoculation since this virus is speeding up its transmission to all over the world, and has limited availability to regions and countries under development.

Vaccination is currently the only method that can effectively stop the spread of SARS-CoV-2 worldwide. Conventional platforms for SARS-CoV-2 vaccines have not yet proven effective. In the present invention, we describe a new type of potent SARS-CoV-2 vaccine based on a yeast surface display system.

### SUMMARY OF THE INVENTION

The present invention provides nucleic acids that permit the expression of SARS-CoV-2 antigens on the surface of a yeast cell, which can then be incorporated into vaccine formulations and used to stimulate an immune response against those antigens.

Accordingly, in one aspect, the invention relates to a nucleic acid construct for heterologous expression of a SARS-CoV-2 antigen on the surface of a yeast cell, the nucleic acid construct including a promoter element active in a yeast cell, operably linked to at least one heterologous polynucleotide that encodes both at least one SARS-CoV-2 antigen and a yeast surface display polypeptide or fragment thereof. The yeast surface display polypeptide is a protein that is part of a yeast surface display system, such as the a-agglutinin (Aga1/Aga2) system or the α-agglutinin system. In certain embodiments, the fragment encoded by the heterologous polynucleotide is an intact subunit of a yeast surface display polypeptide, such as the Aga2 subunit.

In some embodiments, the SARS-CoV-2 antigen is from the SARS-CoV-2 spike protein (“S protein”) and may be, for example, at least 75% identical, at least 80% identical, at least 85% identical, at least 90% identical, at least 95% identical, at least 98% identical, at least 99% identical, or 100% identical to the amino acid sequence set forth in SEQ ID NO: 4.

In some embodiments, the SARS-CoV-2 antigen is specifically from the S1 subunit of the SARS-CoV-2 S protein (“S1 subunit” or “S1 protein”) and may be, for example, at least 75% identical, at least 80% identical, at least 85% identical, at least 90% identical, at least 95% identical, at least 98% identical, at least 99% identical, or 100% identical to the amino acid sequence set forth in SEQ ID NO: 5.

In some embodiments, the SARS-CoV-2 antigen is from the receptor binding domain (“RBD”) of the S1 subunit and may be, for example, at least 75% identical, at least 80% identical, at least 85% identical, at least 90% identical, at least 95% identical, at least 98% identical, at least 99% identical, or 100% identical to the amino acid sequence set forth in SEQ ID NO: 6.

For example, in some embodiments, the heterologous polynucleotide incorporates a nucleotide sequence at least 70%, at least 80%, at least 90%, at least 95%, at least 98%, at least 99%, or 100% identical to a nucleotide sequence as set forth in SEQ ID NO: 1, SEQ ID NO: 2, or SEQ ID NO: 3.

In some embodiments, the yeast surface display polypeptide is an a-agglutinin polypeptide or a fragment thereof. For example, in some embodiments the yeast surface display polypeptide or fragment thereof is an Aga2 peptide, such as a peptide having the amino acid sequence set forth in SEQ ID NO: 7.

Any of the heterologous polynucleotides described above can further encode, for example, a signal peptide in-frame with the SARS-CoV-2 antigen, such as an Aga2 signal peptide, and/or a coding sequence for a linker peptide, such as a linker peptide having the amino acid sequence (GGGGS)_{N}, wherein N is between 1 and 10, inclusive (e.g. GGGGSGGGGSGGGGS, if N is 3).

In some embodiments, the heterologous polynucleotide further encodes one or more tags, such as a 6×His tag or an epitope tag, such as a V5 epitope tag (e.g. a tag having the amino acid sequence set forth in SEQ ID NO: 9).

In certain embodiments, the heterologous polynucleotide encodes a polypeptide having the yeast surface display polypeptide at one terminus; the other terminus may have, for example, a tag, a signal peptide, or the SARS-CoV-2 antigen. For example, in some embodiments, the polypeptide has a signal peptide at the other terminus, and cleavage of the signal peptide results in a mature polypeptide having the SARS-CoV-2 antigen at that terminus. In some embodiments the yeast surface display polypeptide is at the C-terminus of the polypeptide encoded by the heterologous polynucleotide; in other embodiments, the yeast surface display polypeptide is at the N-terminus.

For example, in some embodiments the heterologous polynucleotide encodes a polypeptide containing, in the N→C direction: an Aga2 signal peptide; at least one SARS-CoV-2 antigen; a linker peptide sequence; and an Aga2 peptide. Cleavage of the Aga2 signal peptide results in a mature polypeptide having the SARS-CoV-2 antigen at the N terminus. In certain embodiments, the linker peptide sequence has the amino acid sequence (GGGGS)_{N}, wherein N is between 1 and 10, inclusive (e.g., 3). In some embodiments, the Aga2 peptide at the C-terminus has the amino acid sequence set forth in SEQ ID NO: 7. In some embodiments, the nucleic acid construct incorporates a majority of plasmid pYD5 (Wang et al., 2005, “A new yeast display vector permitting free scFv amino termini can augment ligand binding affinities,” Protein Engineering, Design & Selection, vol. 18(7) pp. 337-343).

In other embodiments, the heterologous polynucleotide encodes a polypeptide comprising, in the N→C direction: as Aga2 signal peptide, an Aga2 peptide, a linker peptide, and at least one SARS-CoV-2 antigen. Cleavage of the Aga2 signal peptide results in a mature polypeptide having the Aga2 peptide at the N-terminus. In some embodiments, the polypeptide includes at least one tag (such as a V5 epitope tag or a 6×His tag) located C-terminal to the at least one SARS-CoV-2 antigen. In some embodiments, the linker peptide has the amino acid sequence (GGGGS)_{N}, wherein N is between 1 and 10, inclusive (e.g., 3). In some embodiments, the Aga2 peptide has the amino acid sequence set forth in SEQ ID NO: 7. In some embodiments, the nucleic acid construct incorporates a majority of plasmid pYD1 (Wang et al., 2005).

For example, in some embodiments, the nucleic acid construct comprises: (a) a promoter element active in a yeast cell; and (b) one or more heterologous polynucleotides encoding (i) a SARS-CoV-2 antigen; and (ii) a yeast surface display polypeptide or fragment thereof, wherein the one or more heterologous polynucleotides are operably linked to the promoter element, and wherein the one or more heterologous polynucleotides encodes a polypeptide comprising, in the N→C direction: (1) an Aga2 signal peptide having the amino acid sequence of SEQ ID NO: 10; (2) the SARS-CoV-2 antigen; and (3) a polypeptide having the amino acid sequence of SEQ ID NO: 12. In some embodiments, the one or more heterologous polynucleotides encodes a polypeptide consisting of, in the N→C direction: (1) an Aga2 signal peptide having the amino acid sequence of SEQ ID NO: 10; (2) the SARS-CoV-2 antigen; and (3) a polypeptide having the amino acid sequence of SEQ ID NO: 12. In some embodiments, the SARS-CoV-2 antigen comprises a sequence selected from the group consisting of SEQ ID NO: 4, SEQ ID NO: 5, and SEQ ID NO: 6.

In some embodiments, the nucleic acid construct comprises: (a) a promoter element active in a yeast cell; and (b) one or more heterologous polynucleotides encoding (i) a SARS-CoV-2 antigen; and (ii) a yeast surface display polypeptide or fragment thereof, wherein the one or more heterologous polynucleotides are operably linked to the promoter element, and wherein the one or more heterologous polynucleotides encodes a polypeptide comprising a sequence as set forth in SEQ ID NO: 13. In some embodiments, the one or more heterologous polynucleotides encodes a polypeptide consisting of a sequence as set forth in SEQ ID NO: 13. In some embodiments, the one or more heterologous polynucleotides comprises a sequence at least 99% identical to SEQ ID NO: 20.

In some embodiments, the nucleic acid comprises: (a) a promoter element active in a yeast cell; and (b) one or more heterologous polynucleotides encoding (i) a SARS-CoV-2 antigen; and (ii) a yeast surface display polypeptide or fragment thereof, wherein the one or more heterologous polynucleotides are operably linked to the promoter element, and wherein the one or more heterologous polynucleotides encodes a polypeptide comprising a sequence as set forth in SEQ ID NO: 14. In some embodiments, the one or more heterologous polynucleotides encodes a polypeptide consisting of a sequence as set forth in SEQ ID NO: 14. In some embodiments, the one or more heterologous polynucleotides comprises a sequence at least 99% identical to SEQ ID NO: 21.

In some embodiments, the nucleic acid construct comprises: (a) a promoter element active in a yeast cell; and (b) one or more heterologous polynucleotides encoding (i) a SARS-CoV-2 antigen; and (ii) a yeast surface display polypeptide or fragment thereof, wherein the one or more heterologous polynucleotides are operably linked to the promoter element, and wherein the one or more heterologous polynucleotides encodes a polypeptide comprising a sequence as set forth in SEQ ID NO: 15. In some embodiments, the one or more heterologous polynucleotides encodes a polypeptide consisting of a sequence as set forth in SEQ ID NO: 15. In some embodiments, the one or more heterologous polynucleotides comprises a sequence at least 99% identical to SEQ ID NO: 22.

In another aspect, the invention relates to a polypeptide expressed by a nucleic acid construct disclosed herein, or a mature version (e.g., after removal of any signal peptides) of such a polypeptide. In some embodiments, the polypeptide comprises, in the N→C direction, an Aga2 signal peptide, a SARS-CoV-2 antigen, a V5 epitope, a (G₄S)_{N} linker, and an Aga2 subunit, wherein N is between 1 and 10. In some embodiments, the polypeptide comprises, in the N→C direction, a SARS-CoV-2 antigen, a V5 epitope, a (G₄S)_{N} linker, and an Aga2 subunit, wherein N is between 1 and 10. In some embodiments, N is 3.

In another aspect, the invention relates to a recombinant yeast containing any of the above-described nucleic acid constructs or any of the above-described polypeptides. In some embodiments, the recombinant yeast also includes a nucleic acid encoding a second yeast surface display polypeptide, which is optionally operably linked to an inducible promoter. In some embodiments, the second yeast surface display polypeptide is an a-agglutinin polypeptide or fragment thereof, such as an Aga1 peptide. In some embodiments, the yeast is S. cerevisiae, such as strain EBY100. In preferred embodiments, the recombinant yeast displays the one or more SARS-CoV-2 antigens encoded by the heterologous polynucleotide on its cell surface. The SARS-CoV-2 antigens can be from, for example, the SARS-CoV-2 S protein, the SARS-CoV-2 S1 subunit, and/or the SARS-CoV-2 RBD.

In another aspect, the invention provides a vaccine composition that includes an effective amount of any of these recombinant yeasts, or an extract thereof. In some embodiments, the vaccine composition includes an adjuvant; in other embodiments, it does not.

The invention also provides, in another aspect, oral dosage formulations of these vaccine compositions. In some embodiments, the oral dosage formulations include an oral solid dosage form excipient, such as a binder, filler, coating, lubricant, matrix-former, and/or disintegrant. In other embodiments, the oral dosage formulation is a liquid or gel formulation, which may optionally be in a monophasic form, such as an aqueous or non-aqueous solution, or a biphasic form, such as a suspension, emulsion, or mixture.

In another aspect, the invention provides methods of inducing an antigen-specific immune response to SARS-CoV-2 in a subject by administering an effective amount of one of the vaccine compositions or oral dosage formulations. The subject may not have tested positive for SARS-CoV-2, and may have tested negative. In preferred embodiments, the subject is human, although other animals susceptible to contracting the virus could also receive the vaccine compositions or oral dosage formulations. The administration is preferably oral administration.

Thus, oral vaccines for preventing SARS CoV-2 infection in humans are described. For example, N-terminal display plasmid pYD5 can be modified to display SARS CoV-2 S, S1, or RBD protein on the surface of S. cerevisiae strain EBY100, and their expression can be detected by Western blotting, immunofluorescence, and/or flow cytometric assay. In some embodiments, the recombinant yeast is mixed with pellets for oral delivery, followed by evaluation of immune response.

In another aspect, the present invention provides a number of useful processes, including: (i) the construction of recombinant yeast; (ii) the mixing of the recombinant yeast with feeding pellets; and (iii) the evaluation of pellet-fed subjects in terms of humoral and cellular immune response.

### BRIEF DESCRIPTION OF THE DRAWINGS

FIG. 1 is a schematic showing the elements of a polypeptide expressed from a nucleic acid construct disclosed herein. The depicted construct is based on a pYD5 vector. Amino acid sequences for the Aga2 signal peptide (SEQ ID NO: 10), V5 epitope (SEQ ID NO: 9), (G₄S)₃ linker (SEQ ID NO: 12), and Aga2 mature peptide (SEQ ID NO: 7) are shown.

FIG. 2 depicts a yeast surface display system that includes a mature polypeptide expressed from a nucleic construct disclosed herein. The mature polypeptide comprises a Aga2 subunit of a-agglutinin, which interacts via disulfide bridges with the Aga1 a-agglutinin subunit expressed on the surface of a yeast cell, such as the EBY100 cell.

### DETAILED DESCRIPTION OF INVENTION

Oral administration is a convenient and effective way to deliver a SARS CoV-2 vaccine and to increase the rate of inoculation. Oral vaccines will be more acceptable because of their oral rather than injectable route of administration with reduced risk of cross-contamination, their avoidance of needle injury, and their increased acceptability to the public in general and to children in particular. Indeed, producing the vaccines in plants could reduce the cost to less than a penny per dose, and simple fast food processing like drying and grinding could create non-perishable preparations without refrigeration.

Adaptive immunity is the key for efficacy of vaccine-induced immune response although innate immunity is also an essential component of the human immune system. Vaccination-induced pathogen-specific memory B and T lymphocytes, as well as functional molecules produced such as antibodies and cytokines, play important roles against invading bacterial, fungal and viral pathogens in humans.

Although the mechanism underlying the interaction between SARS-CoV-2 and host cells needs further investigation, S protein (134.36 KDa) (in particular the S1 subunit (76.5 KDa) of the S protein, and even more in particular the receptor binding domain (RBD) (35.1 KDa) of the S1 subunit) appears to be a major surface antigenic protein of SARS-CoV-2 and involved in the infection process as an attachment protein. Thus, the S protein (in particular the S1 subunit and even more in particular RBD of the S1 subunit) of SARS-CoV-2 may contain preferred antigens for expression in the yeast surface expression system described herein.

### Nucleic Acid Constructs

SARS-CoV-2 Antigens

As used herein, a “SARS-CoV-2 antigen” refers to a SARS-CoV-2 protein or fragment thereof that is capable of eliciting an immune response, e.g., an antibody response, when introduced into a subject having an adaptive immune system (e.g., a subject such as a mammal, e.g., a human). SARS-CoV-2 antigens may vary in length ranging from short peptides (e.g., 8-20 amino acids in length) to longer peptides to full proteins.

The S (spike) protein of SARS-CoV-2 mediates entry of the virus into cells. The S protein is cleaved into the S1 and S2 subunits. The S1 subunit contains a receptor-binding domain (RBD) that specifically recognizes angiotensin-converting enzyme 2 (ACE2), SARS-CoV-2's receptor on cells.

In some embodiments, the SARS-CoV-2 antigen is from (e.g., contained within) the SARS-CoV-2 S protein. In some such embodiments, the SARS-CoV-2 antigen is from the S1 subunit of the S protein. In some embodiments, or from the receptor binding domain (RBD) of the S1 subunit.

Yeast Surface Display Polypeptides

As used herein, a “yeast surface display polypeptide” is a polypeptide that forms part of a yeast surface display system. Yeast surface display systems generally comprise at least one protein or protein subunit that serves as an anchor on the yeast cell wall. In some embodiments, the anchor is fused directly or indirectly to the SARS-CoV-2 antigen. In some embodiments, the anchor interacts with another protein or protein subunit of the yeast surface display system, and the other protein or protein subunit is fused directly or indirectly to the SARS-CoV-2 antigen. This interaction may be, for example, a covalent interaction (e.g., via one or more disulfide bridges) or non-covalent interaction (e.g., binding).

Thus, in many embodiments, the heterologous polynucleotide encodes a polypeptide in which the SARS-CoV-2 antigen is fused to the yeast surface display polypeptide directly or via indirectly (e.g., with one or more intervening elements, such as a linker and/or a tag).

Non-limiting examples of yeast surface display systems include proteins that are members of glycosylphosphatidylinositol (GPI)-anchored proteins and members of the proteins with internal repeats (PIR) family. Thus, for example, suitable yeast surface display systems include, but are not limited to, agglutinin systems (e.g., an a-agglutinin (Aga1p/Aga2p)), Dan4p systems, Sed1p systems, flocculin systems (e.g., comprising Flo1p), cell wall protein systems (e.g., comprising Cw1p, Cwp2p, or Tip1p), and PIR-based systems (e.g., comprising Pir1p Pir2p, Pir3p, Pir4p, or Pir5p).

In some embodiments, the yeast surface display polypeptide is part of the a-agglutinin system, e.g., the yeast surface display polypeptide may be the Aga2 subunit. FIG. 2 shows a schematic of an exemplary yeast surface display system displaying a SARS-CoV-2 antigen.

Fragments of yeast surface polypeptides, e.g., functional fragments, may also be used. By “functional” for the purposes of the yeast display system, it is meant that the fragment either (1) is capable of being anchored into the yeast cell wall or (2) is capable of interacting with a polypeptide or polypeptide fragment that is capable of being anchored into the yeast cell wall. In some embodiments, a fragment comprises a functional subunit of a protein.

### Polypeptides

Also disclosed are polypeptides that are expressed by nucleic acid constructs disclosed herein (e.g., after introduction into a yeast cell) or a mature version thereof. For example, a polypeptide may be expressed in an immature form comprising one or more components (e.g., signal peptides) whose removal (e.g., by cleavage) results in a mature form of the polypeptide. Provided polypeptides generally comprise at least a SARS-CoV-2 antigen and a yeast surface display polypeptide. One or more additional elements (e.g., tags, linkers, etc.) may also be included. In some such embodiments, one or more additional elements intervene between the SARS-CoV-2 antigen and the surface display polypeptide. In some embodiments, after maturation (such as cleavage of a signal peptide), the SARS-CoV-2 antigen is located at one terminus (e.g., the N-terminal end or the C-terminal end) of the polypeptide.

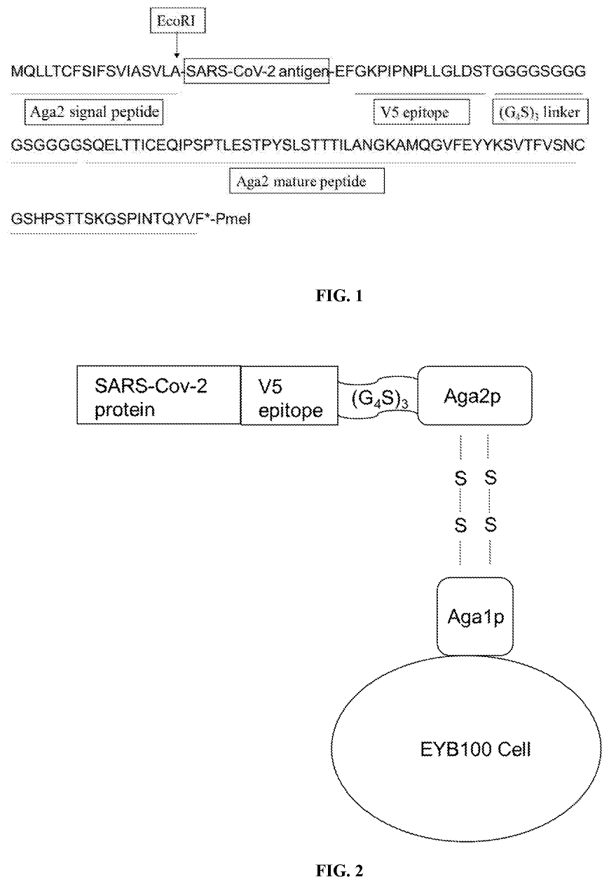

FIG. 1 shows the schematic of an exemplary polypeptide before maturation (e.g., including a signal peptide).

### Recombinant Yeast

Methods of introducing nucleic acid constructs into host yeast strains, thereby generating recombinant yeast strains, are known in the art.

Recombinant yeast comprising a nucleic acid construct or polypeptide as disclosed herein may be generated using any of a variety of yeast strains, including, for example, S. cerevisiae strains and methylotropic strains such as Pichia pastoris and Hansenula polymorpha strains. In some embodiments, the yeast strain either naturally expresses or is engineered to express a second yeast surface display polypeptide (that is, in addition to the yeast surface display polypeptide). The second yeast surface display polypeptide may interact (e.g., covalently or non-covalently bind) to a first yeast surface display polypeptide expressed from a nucleic acid construct disclosed herein. In some embodiments, the second yeast surface display polypeptide is inducibly expressed, e.g., by way of being operably linked to an inducible promoter such as a GAL promoter. For example, the yeast strain may be the S. cerevisiae EBY100 strain, which has a genomic insertion of a gene encoding Aga1 (a component of the agglutinin yeast surface display system) regulated by the Gal promoter with a URA3 selectable marker.

### Vaccine Compositions

Provided vaccine compositions comprise a recombinant yeast cell as described herein, and/or an extract thereof. Methods of preparing yeast extracts are known in the art, and such methods generally involve causing degrading of the cell wall. For example, some methods comprise heating a suspension of a recombinant yeast cell, which may lead to yeast enzymes degrading the cell wall.

Extracts included in a provided vaccine compositions generally comprise a polypeptide comprising a SARS-CoV-2 antigen or a fragment thereof. Polypeptides expressed from nucleic acid constructs disclosed herein may or may not be fragmented, at least partially denatured, or otherwise altered during the process of extraction. Such altered polypeptides may nevertheless function as components of vaccine compositions in that they may nevertheless elicit immune responses.

In some embodiments, provided vaccine compositions comprise one or more adjuvants, substances which accelerate, enhance, and/or prolong immune responses triggered by antigens. A variety of adjuvants known in the art are suitable for use in mammals including humans.

In some embodiments, the composition lacks an adjuvant.

### Oral Dosage Formulations

Provided oral dosage formulations include both solid and non-solid (e.g., liquid or gel) formulations. Generally, in addition to a vaccine composition provided herein, provided formulations include one or more excipients. Suitable excipients for solid oral dosage formulations are known in the art, and include, for example, binders and fillers, coatings, lubricants, matrix formers, and disintegrants.

Liquid or gel formulations may be in aqueous or non-aqueous form, and, for example, in monophasic form or biphasic form (e.g., suspensions, emulsions, and mixtures).

In some embodiments, oral dosage formulations include one or more flavoring agents.

### Methods of Inducing an Antigen-Specific Immune Response to SARS-CoV-2

Provided methods generally comprise a step of administering an effective amount of a vaccine composition or an oral dosage formulation to a subject. By “effective amount” is meant an amount sufficient to effect beneficial or desired results, such as an antigen-specific immune response. An “effective amount” depends upon the context in which it is being applied. An effective amount may be administered by administering a single dose or multiple (e.g., at least two, or at least three) doses.

In some embodiments, the step of administering comprises administering a single dose without administering additional doses. In some embodiments, the step of administering comprises administering an initial dose followed by one or more booster doses.

The subject may be, e.g., a mammal (such as a human or non-human mammal).

In some embodiments, the subject has no existing immunity against SARS-CoV-2 at the time of administration.

In some embodiments, the subject has some existing immunity to SARS-CoV-2. In some embodiments, the existing immunity is deemed insufficient.

In some embodiments, the status of the subject's immunity to SARS-CoV-2 is unknown.

In some embodiments, the subject has not tested positive for SARS-CoV-2 (e.g., the subject has not tested positive in an RNA test intended to identify active infections). In some embodiments, the subject has not tested positive for antibodies against SARS-CoV-2 (e.g., the subject has not tested positive for antibodies against SARS-CoV-2).

In some embodiments, the subject has previously tested positive either for SARS-CoV-2 (e.g., SARS-CoV-2 RNA) or for antibodies against SARS-CoV-2. Administration in these contexts may, for example, provide an immune response to SARS-CoV-2 (if such a response was not previously elicited by exposure to SARS-CoV-2) or to boost a weak (or waned) immune response.

### EXEMPLIFICATION

### Construction of COV-2 Antigen Surface-Displayed Yeast Vaccines

The S protein gene (NCBI MN908947) is PCR-amplified using specific primers and subcloned into pYD5 in-frame with the endogenous Aga2p signal peptide sequence. The resultant shuttle plasmid pYD5-S is transformed into E. coli DH5a. Plasmid pYD5-S is then extracted from E. coli, purified and electroporated into competent S. cerevisiae EBY100 after being linearized. Recombinant yeast transformants are plated on selective minimal dextrose plates containing amino acids (0.67% yeast nitrogen base without amino acids (YNB), 2% glucose, 0.01% leucine, 2% agar, and 1 M sorbitol). Trp⁺ transformants are selected after 3 days of growth on the selective minimal dextrose plates.

Positive colonies are confirmed by genomic PCR. Recombinant S. cerevisiae EBY100/pYD5-S is cultured in YNB-CAA-Glu (0.67% YNB, 0.5 casamino acids, 2% Glucose) and induced in YNB-CAA-Gal (0.67% YNB, 0.5 casamino acids, 2% Galactose, 13.61 g/L Na₂HPO₄, 7.48 g/L NaH₂PO₄ and 5 g/L casamino acids) at 20° C. with shaking (250 rpm) for inducing S surface display. S. cerevisiae EBY 100 carrying pYD5 plasmid serve as a negative control for these tests.

Two additional types of vaccines are constructed in this section: S. cerevisiae EBY100/pYD5-S1 surface displayed yeast vaccine S. cerevisiae EBY100/pYD5-RBD surface displayed yeast vaccine.

### Determining the Functional Display of SARS CoV-2 Antigen on Yeast Surface

These experiments are designed to validate the functional display of the SARS CoV-2 antigen on yeast surface.

Western Blotting

1 OD₆₀₀(1 OD₆₀₀≈10⁷ cells) equivalent recombinant yeast are collected at different time points post inducement with 2% galactose. Samples are washed three times with 500 μl of PBS, re-suspended in 50 μl of 6×SDS loading buffer (Bio-Rad, Hercules, Calif.), and boiled for 10 min. Surface-presented S protein are extracted by heating 1 OD₆₀₀ of S. cerevisiae EBY100/pYD5-S pellets at 95° C. in a Bromophenol blue sample buffer supplemented with 5%-ME for 5 min. Samples are then resolved on a 4-15% SDS-PAGE gel (Bio-Rad), and transferred to 0.45 um nitrocellulose membranes (Bio-Rad). After blocking with 5% non-fat milk at room temperature for 2 h, membranes are incubated with monoclonal mouse anti-S antibody (Sino Biological, Beijing, China) as primary antibody (1:500 diluted). After incubation overnight at 4° C. and washed three times using PBS buffer, the membranes are reacted to secondary antibodies, horseradish peroxidase (HRP)-conjugated rabbit anti-mouse IgG (1:5,000 diluted) (Sigma-Aldrich Co., St. Louis, Mo.) for 1 hour at room temperature. Signals are generated using West Pico chemiluminescent substrates (Thermo Fisher Scientific Inc., Rockford, Ill.) and detected using a ChemiDoc XRS System (Bio-Rad).

Similar methods may be used for the following yeast vaccines S. cerevisiae EBY100/pYD5-S1 and -RBD.

Glycosylation Analysis of One or More Yeast Surface Displayed COV-2 Antigens

PNGase F is obtained from New England Labs (Beverly, Mass.). Recombinant S. cerevisiae EBY100/pYD5-S, S. cerevisiae EBY100/pYD5-S1, or S. cerevisiae EBY100/pYD5-RBD are cultured at 30° C. in YNB-CAA-Glu overnight and then induced at 20° C. in YNB-CAA-Gal for 72 hours. 1 OD₆₀₀ equivalent cells are collected, centrifuged, and washed once in a PBS buffer. Cell pellets are denatured at 100° C. for 10 min in a denaturing buffer included in the PNGase F reagent. A portion of 1 μL of PNGase F (5,000 U) is added to the denatured protein solution, followed by incubation at 37° C. for 1 hour according to the manufacturer's instruction. The treated samples are then be subjected to Western blotting analysis.

Immunofluorescence Microscopy

To detect S display on yeast surface, recombinant S. cerevisiae EBY100/pYD5-S are collected in a 24-hour interval over a 72-hour time period after inducement with galactose (2%). 1 OD₆₀₀ equivalent recombinant yeast is collected and blocked with 5% non-fat milk in PBS for 1 hour, and incubated with monoclonal mouse anti-S antibodies (1:500 diluted) at 4° C. for 1 hour. After washing with PBS, samples are incubated with rabbit anti-mouse IgG FITC conjugates (Sigma) (1:5,000 diluted) at room temperature for 1 h. Samples are kept in the dark until use. FITC-labeled yeast are examined under an inverted phase contrast fluorescence microscope.

To detect S1 display on yeast surface, recombinant S. cerevisiae EBY100/pYD5-S1 cells are collected in a 24-hour interval over a 72-hour time period after inducement with galactose (2%). 1 OD₆₀₀ equivalent recombinant yeast are collected and blocked with 5% non-fat milk in PBS for 1 hour, and incubated with monoclonal mouse anti-S1 antibodies (1:500 diluted) at 4° C. for 1 hour. After washing with PBS, samples are incubated with rabbit anti-mouse IgG FITC conjugates (Sigma) (1:5,000 diluted) at room temperature for 1 h. Samples are kept in the dark until use. FITC-labeled yeast are examined under an inverted phase contrast fluorescence microscope.

To detect RBD displayed on yeast surface, recombinant S. cerevisiae EBY100/pYD5-RBD are collected in a 24-hour interval over a 72-hour time period after inducement with galactose (2%). 1 OD₆₀₀ equivalent recombinant yeast are collected and blocked with 5% non-fat milk in PBS for 1 hour, and incubated with monoclonal mouse anti-RBD antibodies (1:500 diluted) at 4° C. for 1 hour. After washing with PBS, samples are incubated with rabbit anti-mouse IgG FITC conjugates (Sigma) (1:5,000 diluted) at room temperature for 1 h. Samples are kept in the dark until use. FITC-labeled yeast are examined under an inverted phase contrast fluorescence microscope.

Flow Cytometric Assay

After inducement with galactose (2%), 1 OD₆₀₀ equivalent recombinant yeast cells are collected over a 72-hour time period, with a 24-hour interval, as described above. Cell samples are washed three times with sterile PBS containing 1% bovine serum albumin (BSA) and incubated with monoclonal mouse anti-S antibodies (1:500 diluted) at 4° C. for 1 hour, followed by reacting with FITC-conjugated goat anti-mouse IgG (1:5,000) at 4° C. for 30 min. Cell samples are re-suspended in 500 μL of sterile PBS and are subject to flow cytometric analysis using a BD FACS Aira II (BD Bioscience, San Jose, Calif.). S. cerevisiae EBY100/pYD5 served as a negative control for the assay. These data are used to ascertain time points that are ideal for collecting yeast vaccines that present the highest level of one or more of the SARS-CoV-2 antigens on their surface.

Similar methods are used to determine the functional display for the following yeast vaccines S. cerevisiae EBY100/pYD5-S1 and -RBD.

### Optimization of Oral Immunization

Commercial mouse pellet feed weighing 2 g are coated with 3 ml of 1×10⁸ pfu/ml of recombinant yeast. The feed is mixed and incubated on ice for 30 min followed by room temperature (RT) incubation for 30 min to allow absorption. The pellets are coated with fish oil to prevent dispersion.

Balb/c mice of 25 are divided into 5 groups with 5 in each group. Mice in groups 1-3 are administrated orally with recombinant yeast-S, yeast-S1, or yeast-RBD coated feed, respectively, continuously for 7 days, whereas mice in groups 4-5 are administered orally with PBS or yeast containing empty plasmid coated feed, respectively.

### Assessment of Immune Responses

Two weeks after final vaccination, serum of mice are collected, and antibody against S, S1, and RBD are detected by ELISA. Oral vaccination experiments are repeated three times.

Based on results from these experiments, the strength and degree of immune protection that can be provided by the yeast vaccines can be evaluated. Further, it can be determined which vaccines provide better immune protection from virus challenge in animal models.

SEQUENCES

SEQ ID NO: 1 (S gene sequence)
1-atgtttgt ttttcttgtt ttattgccac tagtctctag
tcagtgtgtt aatcttacaa ccagaactca attaccccct gcatacacta attctttcac
acgtggtgtt tattaccctg acaaagtttt cagatcctca gttttacatt caactcagga
cttgttctta cctttctttt ccaatgttac ttggttccat gctatacatg tctctgggac
caatggtact aagaggtttg ataaccctgt cctaccattt aatgatggtg tttattttgc
ttccactgag aagtctaaca taataagagg ctggattttt ggtactactt tagattcgaa
gacccagtcc ctacttattg ttaataacgc tactaatgtt gttattaaag tctgtgaatt
tcaattttgt aatgatccat ttttgggtgt ttattaccac aaaaacaaca aaagttggat
ggaaagtgag ttcagagttt attctagtgc gaataattgc acttttgaat atgtctctca
gccttttctt atggaccttg aaggaaaaca gggtaatttc aaaaatctta gggaatttgt
gtttaagaat attgatggtt attttaaaat atattctaag cacacgccta ttaatttagt
gcgtgatctc cctcagggtt tttcggcttt agaaccattg gtagatttgc caataggtat
taacatcact aggtttcaaa ctttacttgc tttacataga agttatttga ctcctggtga
ttcttcttca ggttggacag ctggtgctgc agcttattat gtgggttatc ttcaacctag
gacttttcta ttaaaatata atgaaaatgg aaccattaca gatgctgtag actgtgcact
tgaccctctc tcagaaacaa agtgtacgtt gaaatccttc actgtagaaa aaggaatcta
tcaaacttct aactttagag tccaaccaac agaatctatt gttagatttc ctaatattac
aaacttgtgc ccttttggtg aagtttttaa cgccaccaga tttgcatctg tttatgcttg
gaacaggaag agaatcagca actgtgttgc tgattattct gtcctatata attccgcatc
attttccact tttaagtgtt atggagtgtc tcctactaaa ttaaatgatc tctgctttac
taatgtctat gcagattcat ttgtaattag aggtgatgaa gtcagacaaa tcgctccagg
gcaaactgga aagattgctg attataatta taaattacca gatgatttta caggctgcgt
tatagcttgg aattctaaca atcttgattc taaggttggt ggtaattata attacctgta
tagattgttt aggaagtcta atctcaaacc ttttgagaga gatatttcaa ctgaaatcta
tcaggccggt agcacacctt gtaatggtgt tgaaggtttt aattgttact ttcctttaca
atcatatggt ttccaaccca ctaatggtgt tggttaccaa ccatacagag tagtagtact
ttcttttgaa cttctacatg caccagcaac tgtttgtgga cctaaaaagt ctactaattt
ggttaaaaac aaatgtgtca atttcaactt caatggttta acaggcacag gtgttcttac
tgagtctaac aaaaagtttc tgcctttcca acaatttggc agagacattg ctgacactac
tgatgctgtc cgtgatccac agacacttga gattcttgac attacaccat gttcttttgg
tggtgtcagt gttataacac caggaacaaa tacttctaac caggttgctg ttctttatca
ggatgttaac tgcacagaag tccctgttgc tattcatgca gatcaactta ctcctacttg
gcgtgtttat tctacaggtt ctaatgtttt tcaaacacgt gcaggctgtt taataggggc
tgaacatgtc aacaactcat atgagtgtga catacccatt ggtgcaggta tatgcgctag
ttatcagact cagactaatt ctcctcggcg ggcacgtagt gtagctagtc aatccatcat
tgcctacact atgtcacttg gtgcagaaaa ttcagttgct tactctaata actctattgc
catacccaca aattttacta ttagtgttac cacagaaatt ctaccagtgt ctatgaccaa
gacatcagta gattgtacaa tgtacatttg tggtgattca actgaatgca gcaatctttt
gttgcaatat ggcagttttt gtacacaatt aaaccgtgct ttaactggaa tagctgttga
acaagacaaa aacacccaag aagtttttgc acaagtcaaa caaatttaca aaacaccacc
aattaaagat tttggtggtt ttaatttttc acaaatatta ccagatccat caaaaccaag
caagaggtca tttattgaag atctactttt caacaaagtg acacttgcag atgctggctt
catcaaacaa tatggtgatt gccttggtga tattgctgct agagacctca tttgtgcaca
aaagtttaac ggccttactg ttttgccacc tttgctcaca gatgaaatga ttgctcaata
cacttctgca ctgttagcgg gtacaatcac ttctggttgg acctttggtg caggtgctgc
attacaaata ccatttgcta tgcaaatggc ttataggttt aatggtattg gagttacaca
gaatgttctc tatgagaacc aaaaattgat tgccaaccaa tttaatagtg ctattggcaa
aattcaagac tcactttctt ccacagcaag tgcacttgga aaacttcaag atgtggtcaa
ccaaaatgca caagctttaa acacgcttgt taaacaactt agctccaatt ttggtgcaat
ttcaagtgtt ttaaatgata tcctttcacg tcttgacaaa gttgaggctg aagtgcaaat
tgataggttg atcacaggca gacttcaaag tttgcagaca tatgtgactc aacaattaat
tagagctgca gaaatcagag cttctgctaa tcttgctgct actaaaatgt cagagtgtgt
acttggacaa tcaaaaagag ttgatttttg tggaaagggc tatcatctta tgtccttccc
tcagtcagca cctcatggtg tagtcttctt gcatgtgact tatgtccctg cacaagaaaa
gaacttcaca actgctcctg ccatttgtca tgatggaaaa gcacactttc ctcgtgaagg
tgtctttgtt tcaaatggca cacactggtt tgtaacacaa aggaattttt atgaaccaca
aatcattact acagacaaca catttgtgtc tggtaactgt gatgttgtaa taggaattgt
caacaacaca gtttatgatc ctttgcaacc tgaattagac tcattcaagg aggagttaga
taaatatttt aagaatcata catcaccaga tgttgattta ggtgacatct ctggcattaa
tgcttcagtt gtaaacattc aaaaagaaat tgaccgcctc aatgaggttg ccaagaattt
aaatgaatct ctcatcgatc tccaagaact tggaaagtat gagcagtata taaaatggcc
atggtacatt tggctaggtt ttatagctgg cttgattgcc atagtaatgg tgacaattat
gctttgctgt atgaccagtt gctgtagttg tctcaagggc tgttgttctt gtggatcctg
ctgcaaattt gatgaagacg actctgagcc agtgctcaaa ggagtcaaat tacattacac
ataa-3822
SEQ ID NO: 2 (S1 nucleotide sequence)
1-atgtttgt ttttcttgtt ttattgccac tagtctctag
tcagtgtgtt aatcttacaa ccagaactca attaccccct gcatacacta attctttcac
acgtggtgtt tattaccctg acaaagtttt cagatcctca gttttacatt caactcagga
cttgttctta cctttctttt ccaatgttac ttggttccat gctatacatg tctctgggac
caatggtact aagaggtttg ataaccctgt cctaccattt aatgatggtg tttattttgc
ttccactgag aagtctaaca taataagagg ctggattttt ggtactactt tagattcgaa
gacccagtcc ctacttattg ttaataacgc tactaatgtt gttattaaag tctgtgaatt
tcaattttgt aatgatccat ttttgggtgt ttattaccac aaaaacaaca aaagttggat
ggaaagtgag ttcagagttt attctagtgc gaataattgc acttttgaat atgtctctca
gccttttctt atggaccttg aaggaaaaca gggtaatttc aaaaatctta gggaatttgt
gtttaagaat attgatggtt attttaaaat atattctaag cacacgccta ttaatttagt
gcgtgatctc cctcagggtt tttcggcttt agaaccattg gtagatttgc caataggtat
taacatcact aggtttcaaa ctttacttgc tttacataga agttatttga ctcctggtga
ttcttcttca ggttggacag ctggtgctgc agcttattat gtgggttatc ttcaacctag
gacttttcta ttaaaatata atgaaaatgg aaccattaca gatgctgtag actgtgcact
tgaccctctc tcagaaacaa agtgtacgtt gaaatccttc actgtagaaa aaggaatcta
tcaaacttct aactttagag tccaaccaac agaatctatt gttagatttc ctaatattac
aaacttgtgc ccttttggtg aagtttttaa cgccaccaga tttgcatctg tttatgcttg
gaacaggaag agaatcagca actgtgttgc tgattattct gtcctatata attccgcatc
attttccact tttaagtgtt atggagtgtc tcctactaaa ttaaatgatc tctgctttac
taatgtctat gcagattcat ttgtaattag aggtgatgaa gtcagacaaa tcgctccagg
gcaaactgga aagattgctg attataatta taaattacca gatgatttta caggctgcgt
tatagcttgg aattctaaca atcttgattc taaggttggt ggtaattata attacctgta
tagattgttt aggaagtcta atctcaaacc ttttgagaga gatatttcaa ctgaaatcta
tcaggccggt agcacacctt gtaatggtgt tgaaggtttt aattgttact ttcctttaca
atcatatggt ttccaaccca ctaatggtgt tggttaccaa ccatacagag tagtagtact
ttcttttgaa cttctacatg caccagcaac tgtttgtgga cctaaaaagt ctactaattt
ggttaaaaac aaatgtgtca atttcaactt caatggttta acaggcacag gtgttcttac
tgagtctaac aaaaagtttc tgcctttcca acaatttggc agagacattg ctgacactac
tgatgctgtc cgtgatccac agacacttga gattcttgac attacaccat gttcttttgg
tggtgtcagt gttataacac caggaacaaa tacttctaac caggttgctg ttctttatca
ggatgttaac tgcacagaag tccctgttgc tattcatgca gatcaactta ctcctacttg
gcgtgtttat tctacaggtt ctaatgtttt tcaaacacgt gcaggctgtt taataggggc
tgaacatgtc aacaactcat atgagtgtga catacccatt ggtgcaggta tatgcgctag
ttatcagact cagactaatt ctcct-2043
SEQ ID NO: 3 (RBD nucleotide sequence)
988-c ctaatattac
aaacttgtgc ccttttggtg aagtttttaa cgccaccaga tttgcatctg tttatgcttg
gaacaggaag agaatcagca actgtgttgc tgattattct gtcctatata attccgcatc
attttccact tttaagtgtt atggagtgtc tcctactaaa ttaaatgatc tctgctttac
taatgtctat gcagattcat ttgtaattag aggtgatgaa gtcagacaaa tcgctccagg
gcaaactgga aagattgctg attataatta taaattacca gatgatttta caggctgcgt
tatagcttgg aattctaaca atcttgattc taaggttggt ggtaattata attacctgta
tagattgttt aggaagtcta atctcaaacc ttttgagaga gatatttcaa ctgaaatcta
tcaggccggt agcacacctt gtaatggtgt tgaaggtttt aattgttact ttcctttaca
atcatatggt ttccaaccca ctaatggtgt tggttaccaa ccatacagag tagtagtact
ttcttttgaa cttctacatg cacca-1563
SEQ ID NO: 4 (S full length protein sequence)
MFVFLVLLPLVSSQCVNLTTRTQLPPAYTNSFTRGVYYPDKVFRSSVLHSTQDLFLPFFSNVTWFHA
IHVSGTNGTKRFDNPVLPFNDGVYFASTEKSNIIRGWIFGTTLDSKTQSLLIVNNATNVVIKVCEFQ
FCNDPFLGVYYHKNNKSWMESEFRVYSSANNCTFEYVSQPFLMDLEGKQGNFKNLREFVFKNIDGYF
KIYSKHTKHTPINLVRDLPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQTSNFRVQPTESIVRFPNI
TNLCPFGEVFNATRFASVYAWNRKRISNCVADYSVLYNSASFSTFKCYGVSPTKLNDLCFTNVYADS
FVIRGDEVRQIAPGQTGKIADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSYECDIPIG
AGICASYQTQTNSPRRARSVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFAQVKQIYKTPPIKDFGGFN
FSQILPDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSAIGKIQ
DSLSSTASALGKLQDVVNQNAQALNTLVKQLSSNFGAISSVLNDILSRLDKVEAEVQIDRLITGRLQ
SLQTYVTQQLIRAAEIRASANLAATKMSECVLGQSKRVDFCGKGYHLMSFPQSAPHGVVFLHVTYVP
AQEKNFTTAPAICHDGKAHFPREGVFVSNGTHWFVTQRNFYEPQIITTDNTFVSGNCDVVIGIVNNT
VYDPLQPELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEPVLKGVKL
HYT
SEQ ID NO: 5 (S1 full length protein sequence)
MFVFLVLLPLVSSQCVNLTTRTQLPPAYTNSFTRGVYYPDKVFRSSVLHSTQDLFLPFFSNVTWFHA
IHVSGTNGTKRFDNPVLPFNDGVYFASTEKSNIIRGWIFGTTLDSKTQSLLIVNNATNVVIKVCEFQ
FCNDPFLGVYYHKNNKSWMESEFRVYSSANNCTFEYVSQPFLMDLEGKQGNFKNLREFVFKNIDGYF
KIYSKHTKHTPINLVRDLPQGFSALEPLVDLPIGINITRFQTLLALHRSYLTPGDSSSGWTAGAAAY
YVGYLQPRTFLLKYNENGTITDAVDCALDPLSETKCTLKSFTVEKGIYQTSNFRVQPTESIVRFPNI
TNLCPFGEVFNATRFASVYAWNRKRISNCVADYSVLYNSASFSTFKCYGVSPTKLNDLCFTNVYADS
FVIRGDEVRQIAPGQTGKIADYNYKLPDDFTGCVIAWNSNNLDSKVGGNYNYLYRLFRKSNLKPFER
DISTEIYQAGSTPCNGVEGFNCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSYECDIPIG
AGICASYQTQTNSP
SEQ ID NO: 6 (RBD full length protein sequence)
PNITNLCPFGEVFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVVLSFELLHAP
SEQ ID NO: 7 (Aga2 mature peptide)
QELTTICEQIPSPTLESTPYSLSTTTILANGKAMQGVFEYYKSVTFVSNCGSHPSTTSKGSPINTQY
VF
SEQ ID NO: 8 (Aga1 protein)
MTLSFAHFTYLFTILLGLTNIALASDPETILVTITKTNDANGVVTTTVSPALVSTSTIVQAGTTTLY
TTWCPLTVSTSSAAEISPSISYATTLSRFSTLTLSTEVCSHEACPSSSTLPTTTLSVTSKFTSYICP
TCHTTAISSLSEVGTTTVVSSSAIEPSSASIISPVTSTLSSTTSSNPTTTSLSSTSTSPSSTSTSPS
STSTSSSSTSTSSSSTSTSSSSTSTSPSSTSTSSSLTSTSSSSTSTSQSSTSTSSSSTSTSPSSTST
SSSSTSTSPSSKSTSASSTSTSSYSTSTSPSLTSSSPTLASTSPSSTSISSTFTDSTSSLGSSIASS
STSVSLYSPSTPVYSVPSTSSNVATPSMTSSTVETTVSSQSSSEYITKSSISTTIPSFSMSTYFTTV
SGVTTMYTTWCPYSSESETSTLTSMHETVTTDATVCTHESCMPSQTTSLITSSIKMSTKNVATSVST
STVESSYACSTCAETSHSYSSVQTASSSSVTQQTTSTKSWVSSMTTSDEDFNKHATGKYHVTSSGTS
TISTSVSEATSTSSIDSESQEQSSHLLSTSVLSSSSLSATLSSDSTILLFSSVSSLSVEQSPVTTLQ
ISSTSEILQPTSSTAIATISASTSSLSATSISTPSTSVESTIESSSLTPTVSSIFLSSSSAPSSLQT
SVTTTEVSTTSISIQYQTSSMVTISQYMGSGSQTRLPLGKLVFAIMAVACNVIFS
SEQ ID NO: 9 (V5 epitope tag)
GKPIPNPLLGLDST
SEQ ID NO: 10 (Aga2 signal peptide)
MQLLTCFSIFSVIASVLA
SEQ ID NO: 11 ((G₄S)₃ linker)
GGGGSGGGGSGGGGS
SEQ ID NO: 12 (right flank of pYD5-based construct shown in FIG. 1)
EFGKPIPNPLLGLDSTGGGGSGGGGSGGGGSQELTTICEQIPSPTLESTPYSLSTTTILANGKAMQG
VFEYYKSVTFVSNCGSHPSTTSKGSPINTQYVF
SEQ ID NO: 13 (amino acid sequence encoded by the construct shown in
FIG. 1, using full S protein as the SARS-CoV-2 antigen)
MQLLTCFSIFSVIASVLAMFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFLMDLEGKQ
GNFKNLREFVFKNIDGYFKIYSKHTKHTPINLVRDLPQGFSALEPLVDLPIGINITRFQTLLALHRS
YLTPGDSSSGWTAGAAAYYVGYLQPRTFLLKYNENGTITDAVDCALDPLSETKCTLKSFTVEKGIYQ
TSNFRVQPTESIVRFPNITNLCPFGEVFNATRFASVYAWNRKRISNCVADYSVLYNSASFSTFKCYG
VSPTKLNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQFGRDIADTTDAVRDPQTL
EILDITPCSFGGVSVITPGTNTSNQVAVLYQDVNCTEVPVAIHADQLTPTWRVYSTGSNVFQTRAGC
LIGAEHVNNSYECDIPIGAGICASYQTQTNSPRRARSVASQSIIAYTMSLGAENSVAYSNNSIAIPT
NFTISVTTEILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAMQMAYRFNGIGVTQNVLY
ENQKLIANQFNSAIGKIQDSLSSTASALGKLQDVVNQNAQALNTLVKQLSSNFGAISSVLNDILSRL
DKVEAEVQIDRLITGRLQSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDR
LNEVAKNLNESLIDLQELGKYEQYIKWPWYIWLGFIAGLIAIVMVTIMLCCMTSCCSCLKGCCSCGS
CCKFDEDDSEPVLKGVKLHYTEFGKPIPNPLLGLDSTGGGGSGGGGSGGGGSQELTTICEQIPSPTL
ESTPYSLSTTTILANGKAMQGVFEYYKSVTFVSNCGSHPSTTSKGSPINTQYVF
SEQ ID NO: 14 (amino acid sequence encoded by the construct shown in
FIG. 1, using full S1 subunit as the SARS-CoV-2 antigen)
MQLLTCFSIFSVIASVLAMFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVSQPFLMDLEGKQ
GNFKNLREFVFKNIDGYFKIYSKHTKHTPINLVRDLPQGFSALEPLVDLPIGINITRFQTLLALHRS
YLTPGDSSSGWTAGAAAYYVGYLQPRTFLLKYNENGTITDAVDCALDPLSETKCTLKSFTVEKGIYQ
TSNFRVQPTESIVRFPNITNLCPFGEVFNATRFASVYAWNRKRISNCVADYSVLYNSASFSTFKCYG
VSPTKLNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPYRVVVLSF
ELLHAPATVCGPKKSTNLVKNKCVNFNFNGLTGTGVLTESNKKFLPFQQFGRDIADTTDAVRDPQTL
EILDITPCSFGGVSVITPGTNTSNQVAVLYQDVNCTEVPVAIHADQLTPTWRVYSTGSNVFQTRAGC
LIGAEHVNNSYECDIPIGAGICASYQTQTNSPEFGKPIPNPLLGLDSTGGGGSGGGGSGGGGSQELT
TICEQIPSPTLESTPYSLSTTTILANGKAMQGVFEYYKSVTFVSNCGSHPSTTSKGSPINTQYVF
SEQ ID NO: 15 (amino acid sequence encoded by the construct shown in
FIG. 1, using full RBD subunit as the SARS-CoV-2 antigen)
MQLLTCFSIFSVIASVLAPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSYGFQPTNGVGYQPYRVVV
LSFELLHAPEFGKPIPNPLLGLDSTGGGGSGGGGSGGGGSQELTTICEQIPSPTLESTPYSLSTTTI
LANGKAMQGVFEYYKSVTFVSNCGSHPSTTSKGSPINTQYVF
SEQ ID NO: 16 (nucleotide sequence for Aga2 signal peptide)
ATG CAG TTA CTT CGC TGT TTT TCA ATA TTT TCT GTT ATT GCT TCA GTT TTA
GCA
SEQ ID NO: 17 (nucleotide sequence for V5 epitope)
GGT AAG CCT ATC CCT AAC CCT CTC CTC GGT CTC GAT TCT ACG
SEQ ID NO: 18 (nucleotide sequence for linker)
GGT GGT GGT GGT TCT GGT GGT GGT GGT TCT GGT GGT GGT GGT TCT
SEQ ID NO: 19 (nucleotide sequence for Aga2 mature peptide)
CAG GAA CTG ACA ACT ATA TGC GAG CAA ATC CCC TCA CCA ACT TTA GAA TCG
ACG CCG TAC TCT TTG TCA ACG ACT ACT ATT TTG GCC AAC GGG AAG GCA ATG
CAA GGA GTT TTT
SEQ ID NO: 20 (nucleotide sequence for the construct shown in FIG. 1,
using the full S protein as the SARS-CoV-2 antigen)

| | |
|---|---|
| 1 | ATGCAGTTAC TTCGCTGTTT TTCAATATTT TCTGTTATTG CTTCAGTTTT |
| 51 | AGCAatgttt gtttttcttg ttttattgcc actagtctct agtcagtgtg |
| 101 | ttaatcttac aaccagaact caattacccc ctgcatacac taattctttc |
| 151 | acacgtggtg tttattaccc tgacaaagtt ttcagatcct cagttttaca |
| 201 | ttcaactcag gacttgttct tacctttctt ttccaatgtt acttggttcc |
| 251 | atgctataca tgtctctggg accaatggta ctaagaggtt tgataaccct |
| 301 | gtcctaccat ttaatgatgg tgtttatttt gcttccactg agaagtctaa |
| 351 | cataataaga ggctggattt ttggtactac tttagattcg aagacccagt |
| 401 | ccctacttat tgttaataac gctactaatg ttgttattaa agtctgtgaa |
| 451 | tttcaatttt gtaatgatcc atttttgggt gtttattacc acaaaaacaa |
| 501 | caaaagttgg atggaaagtg agttcagagt ttattctagt gcgaataatt |
| 551 | gcacttttga atatgtctct cagccttttc ttatggacct tgaaggaaaa |
| 601 | cagggtaatt tcaaaaatct tagggaattt gtgtttaaga atattgatgg |
| 651 | ttattttaaa atatattcta agcacacgcc tattaattta gtgcgtgatc |
| 701 | tccctcaggg tttttcggct ttagaaccat tggtagattt gccaataggt |
| 751 | attaacatca ctaggtttca aactttactt gctttacata gaagttattt |
| 801 | gactcctggt gattcttctt caggttggac agctggtgct gcagcttatt |
| 851 | atgtgggtta tcttcaacct aggacttttc tattaaaata taatgaaaat |
| 901 | ggaaccatta cagatgctgt agactgtgca cttgaccctc tctcagaaac |
| 951 | aaagtgtacg ttgaaatcct tcactgtaga aaaaggaatc tatcaaactt |
| 1001 | ctaactttag agtccaacca acagaatcta ttgttagatt tcctaatatt |
| 1051 | acaaacttgt gcccttttgg tgaagttttt aacgccacca gatttgcatc |
| 1101 | tgtttatgct tggaacagga agagaatcag caactgtgtt gctgattatt |
| 1151 | ctgtcctata taattccgca tcattttcca cttttaagtg ttatggagtg |
| 1201 | tctcctacta aattaaatga tctctgcttt actaatgtct atgcagattc |
| 1251 | atttgtaatt agaggtgatg aagtcagaca aatcgctcca gggcaaactg |
| 1301 | gaaagattgc tgattataat tataaattac cagatgattt tacaggctgc |
| 1351 | gttatagctt ggaattctaa caatcttgat tctaaggttg gtggtaatta |
| 1401 | taattacctg tatagattgt ttaggaagtc taatctcaaa ccttttgaga |
| 1451 | gagatatttc aactgaaatc tatcaggccg gtagcacacc ttgtaatggt |
| 1501 | gttgaaggtt ttaattgtta ctttccttta caatcatatg gtttccaacc |
| 1551 | cactaatggt gttggttacc aaccatacag agtagtagta ctttcttttg |
| 1601 | aacttctaca tgcaccagca actgtttgtg gacctaaaaa gtctactaat |
| 1651 | ttggttaaaa acaaatgtgt caatttcaac ttcaatggtt taacaggcac |
| 1701 | aggtgttctt actgagtcta acaaaaagtt tctgcctttc caacaatttg |
| 1751 | gcagagacat tgctgacact actgatgctg tccgtgatcc acagacactt |
| 1801 | gagattcttg acattacacc atgttctttt ggtggtgtca gtgttataac |
| 1851 | accaggaaca aatacttcta accaggttgc tgttctttat caggatgtta |
| 1901 | actgcacaga agtccctgtt gctattcatg cagatcaact tactcctact |
| 1951 | tggcgtgttt attctacagg ttctaatgtt tttcaaacac gtgcaggctg |
| 2001 | tttaataggg gctgaacatg tcaacaactc atatgagtgt gacataccca |
| 2051 | ttggtgcagg tatatgcgct agttatcaga ctcagactaa ttctcctcgg |
| 2101 | cgggcacgta gtgtagctag tcaatccatc attgcctaca ctatgtcact |
| 2151 | tggtgcagaa aattcagttg cttactctaa taactctatt gccataccca |
| 2201 | caaattttac tattagtgtt accacagaaa ttctaccagt gtctatgacc |
| 2251 | aagacatcag tagattgtac aatgtacatt tgtggtgatt caactgaatg |
| 2301 | cagcaatctt ttgttgcaat atggcagttt ttgtacacaa ttaaaccgtg |
| 2351 | ctttaactgg aatagctgtt gaacaagaca aaaacaccca agaagttttt |
| 2401 | gcacaagtca aacaaattta caaaacacca ccaattaaag attttggtgg |
| 2451 | ttttaatttt tcacaaatat taccagatcc atcaaaacca agcaagaggt |
| 2501 | catttattga agatctactt ttcaacaaag tgacacttgc agatgctggc |
| 2551 | ttcatcaaac aatatggtga ttgccttggt gatattgctg ctagagacct |
| 2601 | catttgtgca caaaagttta acggccttac tgttttgcca cctttgctca |
| 2651 | cagatgaaat gattgctcaa tacacttctg cactgttagc gggtacaatc |
| 2701 | acttctggtt ggacctttgg tgcaggtgct gcattacaaa taccatttgc |
| 2751 | tatgcaaatg gcttataggt ttaatggtat tggagttaca cagaatgttc |
| 2801 | tctatgagaa ccaaaaattg attgccaacc aatttaatag tgctattggc |
| 2851 | aaaattcaag actcactttc ttccacagca agtgcacttg gaaaacttca |
| 2901 | agatgtggtc aaccaaaatg cacaagcttt aaacacgctt gttaaacaac |
| 2951 | ttagctccaa ttttggtgca atttcaagtg ttttaaatga tatcctttca |
| 3001 | cgtcttgaca aagttgaggc tgaagtgcaa attgataggt tgatcacagg |
| 3051 | cagacttcaa agtttgcaga catatgtgac tcaacaatta attagagctg |
| 3101 | cagaaatcag agcttctgct aatcttgctg ctactaaaat gtcagagtgt |
| 3151 | gtacttggac aatcaaaaag agttgatttt tgtggaaagg gctatcatct |
| 3201 | tatgtccttc cctcagtcag cacctcatgg tgtagtcttc ttgcatgtga |
| 3251 | cttatgtccc tgcacaagaa aagaacttca caactgctcc tgccatttgt |
| 3301 | catgatggaa aagcacactt tcctcgtgaa ggtgtctttg tttcaaatgg |
| 3351 | cacacactgg tttgtaacac aaaggaattt ttatgaacca caaatcatta |
| 3401 | ctacagacaa cacatttgtg tctggtaact gtgatgttgt aataggaatt |
| 3451 | gtcaacaaca cagtttatga tcctttgcaa cctgaattag actcattcaa |
| 3501 | ggaggagtta gataaatatt ttaagaatca tacatcacca gatgttgatt |
| 3551 | taggtgacat ctctggcatt aatgcttcag ttgtaaacat tcaaaaagaa |
| 3601 | attgaccgcc tcaatgaggt tgccaagaat ttaaatgaat ctctcatcga |
| 3651 | tctccaagaa cttggaaagt atgagcagta tataaaatgg ccatggtaca |
| 3701 | tttggctagg ttttatagct ggcttgattg ccatagtaat ggtgacaatt |
| 3751 | atgctttgct gtatgaccag ttgctgtagt tgtctcaagg gctgttgttc |
| 3801 | ttgtggatcc tgctgcaaat ttgatgaaga cgactctgag ccagtgctca |
| 3851 | aaggagtcaa attacattac acataaGGTA AGCCTATCCC TAACCCTCTC |
| 3901 | CTCGGTCTCG ATTCTACGGG TGGTGGTGGT TCTGGTGGTG GTGGTTCTGG |
| 3951 | TGGTGGTGGT TCTCAGGAAC TGACAACTAT ATGCGAGCAA ATCCCCTCAC |
| 4001 | CAACTTTAGA ATCGACGCCG TACTCTTTGT CAACGACTAC TATTTTGGCC |
| 4051 | AACGGGAAGG CAATGCAAGG AGTTTTT |

SEQ ID NO: 21 (nucleotide sequence for the construct shown in FIG. 1,
using the full S1 subunit as the SARS-CoV-2 antigen)

| | |
|---|---|
| 1 | ATGCAGTTAC TTCGCTGTTT TTCAATATTT TCTGTTATTG CTTCAGTTTT |
| 51 | AGCAATGTTT GTTTTTCTTG TTTTATTGCC ACTAGTCTCT AGTCAGTGTG |
| 101 | TTAATCTTAC AACCAGAACT CAATTACCCC CTGCATACAC TAATTCTTTC |
| 151 | ACACGTGGTG TTTATTACCC TGACAAAGTT TTCAGATCCT CAGTTTTACA |
| 201 | TTCAACTCAG GACTTGTTCT TACCTTTCTT TTCCAATGTT ACTTGGTTCC |
| 251 | ATGCTATACA TGTCTCTGGG ACCAATGGTA CTAAGAGGTT TGATAACCCT |
| 301 | GTCCTACCAT TTAATGATGG TGTTTATTTT GCTTCCACTG AGAAGTCTAA |
| 351 | CATAATAAGA GGCTGGATTT TTGGTACTAC TTTAGATTCG AAGACCCAGT |
| 401 | CCCTACTTAT TGTTAATAAC GCTACTAATG TTGTTATTAA AGTCTGTGAA |
| 451 | TTTCAATTTT GTAATGATCC ATTTTTGGGT GTTTATTACC ACAAAAACAA |
| 501 | CAAAAGTTGG ATGGAAAGTG AGTTCAGAGT TTATTCTAGT GCGAATAATT |
| 551 | GCACTTTTGA ATATGTCTCT CAGCCTTTTC TTATGGACCT TGAAGGAAAA |
| 601 | CAGGGTAATT TCAAAAATCT TAGGGAATTT GTGTTTAAGA ATATTGATGG |
| 651 | TTATTTTAAA ATATATTCTA AGCACACGCC TATTAATTTA GTGCGTGATC |
| 701 | TCCCTCAGGG TTTTTCGGCT TTAGAACCAT TGGTAGATTT GCCAATAGGT |
| 751 | ATTAACATCA CTAGGTTTCA AACTTTACTT GCTTTACATA GAAGTTATTT |
| 801 | GACTCCTGGT GATTCTTCTT CAGGTTGGAC AGCTGGTGCT GCAGCTTATT |
| 851 | ATGTGGGTTA TCTTCAACCT AGGACTTTTC TATTAAAATA TAATGAAAAT |
| 901 | GGAACCATTA CAGATGCTGT AGACTGTGCA CTTGACCCTC TCTCAGAAAC |
| 951 | AAAGTGTACG TTGAAATCCT TCACTGTAGA AAAAGGAATC TATCAAACTT |
| 1001 | CTAACTTTAG AGTCCAACCA ACAGAATCTA TTGTTAGATT TCCTAATATT |
| 1051 | ACAAACTTGT GCCCTTTTGG TGAAGTTTTT AACGCCACCA GATTTGCATC |
| 1101 | TGTTTATGCT TGGAACAGGA AGAGAATCAG CAACTGTGTT GCTGATTATT |
| 1151 | CTGTCCTATA TAATTCCGCA TCATTTTCCA CTTTTAAGTG TTATGGAGTG |
| 1201 | TCTCCTACTA AATTAAATGA TCTCTGCTTT ACTAATGTCT ATGCAGATTC |
| 1251 | ATTTGTAATT AGAGGTGATG AAGTCAGACA AATCGCTCCA GGGCAAACTG |
| 1301 | GAAAGATTGC TGATTATAAT TATAAATTAC CAGATGATTT TACAGGCTGC |
| 1351 | GTTATAGCTT GGAATTCTAA CAATCTTGAT TCTAAGGTTG GTGGTAATTA |
| 1401 | TAATTACCTG TATAGATTGT TTAGGAAGTC TAATCTCAAA CCTTTTGAGA |
| 1451 | GAGATATTTC AACTGAAATC TATCAGGCCG GTAGCACACC TTGTAATGGT |
| 1501 | GTTGAAGGTT TTAATTGTTA CTTTCCTTTA CAATCATATG GTTTCCAACC |
| 1551 | CACTAATGGT GTTGGTTACC AACCATACAG AGTAGTAGTA CTTTCTTTTG |
| 1601 | AACTTCTACA TGCACCAGCA ACTGTTTGTG GACCTAAAAA GTCTACTAAT |
| 1651 | TTGGTTAAAA ACAAATGTGT CAATTTCAAC TTCAATGGTT TAACAGGCAC |
| 1701 | AGGTGTTCTT ACTGAGTCTA ACAAAAAGTT TCTGCCTTTC CAACAATTTG |
| 1751 | GCAGAGACAT TGCTGACACT ACTGATGCTG TCCGTGATCC ACAGACACTT |
| 1801 | GAGATTCTTG ACATTACACC ATGTTCTTTT GGTGGTGTCA GTGTTATAAC |
| 1851 | ACCAGGAACA AATACTTCTA ACCAGGTTGC TGTTCTTTAT CAGGATGTTA |
| 1901 | ACTGCACAGA AGTCCCTGTT GCTATTCATG CAGATCAACT TACTCCTACT |
| 1951 | TGGCGTGTTT ATTCTACAGG TTCTAATGTT TTTCAAACAC GTGCAGGCTG |
| 2001 | TTTAATAGGG GCTGAACATG TCAACAACTC ATATGAGTGT GACATACCCA |
| 2051 | TTGGTGCAGG TATATGCGCT AGTTATCAGA CTCAGACTAA TTCTCCTGGT |
| 2101 | AAGCCTATCC CTAACCCTCT CCTCGGTCTC GATTCTACGG GTGGTGGTGG |
| 2151 | TTCTGGTGGT GGTGGTTCTG GTGGTGGTGG TTCTCAGGAA CTGACAACTA |
| 2201 | TATGCGAGCA AATCCCCTCA CCAACTTTAG AATCGACGCC GTACTCTTTG |
| 2251 | TCAACGACTA CTATTTTGGC CAACGGGAAG GCAATGCAAG GAGTTTTT |

SEQ ID NO: 22 (nucleotide sequence for the construct shown in FIG. 1,
using the full RBD as the SARS-CoV-2 antigen)

| | |
|---|---|
| 1 | ATGCAGTTAC TTCGCTGTTT TTCAATATTT TCTGTTATTG CTTCAGTTTT |
| 51 | AGCACTAATA TTACAAACTT GTGCCCTTTT GGTGAAGTTT TTAACGCCAC |
| 101 | CAGATTTGCA TCTGTTTATG CTTGGAACAG GAAGAGAATC AGCAACTGTG |
| 151 | TTGCTGATTA TTCTGTCCTA TATAATTCCG CATCATTTTC CACTTTTAAG |
| 201 | TGTTATGGAG TGTCTCCTAC TAAATTAAAT GATCTCTGCT TTACTAATGT |
| 251 | CTATGCAGAT TCATTTGTAA TTAGAGGTGA TGAAGTCAGA CAAATCGCTC |
| 301 | CAGGGCAAAC TGGAAAGATT GCTGATTATA ATTATAAATT ACCAGATGAT |
| 351 | TTTACAGGCT GCGTTATAGC TTGGAATTCT AACAATCTTG ATTCTAAGGT |
| 401 | TGGTGGTAAT TATAATTACC TGTATAGATT GTTTAGGAAG TCTAATCTCA |
| 451 | AACCTTTTGA GAGAGATATT TCAACTGAAA TCTATCAGGC CGGTAGCACA |
| 501 | CCTTGTAATG GTGTTGAAGG TTTTAATTGT TACTTTCCTT TACAATCATA |
| 551 | TGGTTTCCAA CCCACTAATG GTGTTGGTTA CCAACCATAC AGAGTAGTAG |
| 601 | TACTTTCTTT TGAACTTCTA CATGCACCAG GTAAGCCTAT CCCTAACCCT |
| 651 | CTCCTCGGTC TCGATTCTAC GGGTGGTGGT GGTTCTGGTG GTGGTGGTTC |
| 701 | TGGTGGTGGT GGTTCTCAGG AACTGACAAC TATATGCGAG CAAATCCCCT |
| 751 | CACCAACTTT AGAATCGACG CCGTACTCTT TGTCAACGAC TACTATTTTG |
| 801 | GCCAACGGGA AGGCAATGCA AGGAGTTTTT |

### Numbered Embodiments

1. A nucleic acid construct for heterologous expression of a SARS CoV-2 antigen on the surface of a yeast cell, the nucleic acid construct comprising:

(a) a promoter element active in a yeast cell; and

(b) one or more heterologous polynucleotides encoding (i) a SARS-CoV-2 antigen; and (ii) a yeast surface display polypeptide or fragment thereof,

wherein the one or more heterologous polynucleotides are operably linked to the promoter element, and

wherein the one or more heterologous polynucleotides encodes a polypeptide comprising, in the N→C direction:
(1) an Aga2 signal peptide having the amino acid sequence of SEQ ID NO: 10;
(2) the SARS-CoV-2 antigen; and
(3) a polypeptide having the amino acid sequence of SEQ ID NO: 12.
   2. The nucleic acid construct of embodiment 1, wherein the one or more heterologous polynucleotides encodes a polypeptide consisting of, in the N→C direction:
(1) an Aga2 signal peptide having the amino acid sequence of SEQ ID NO: 10;
(2) the SARS-CoV-2 antigen; and
(3) a polypeptide having the amino acid sequence of SEQ ID NO: 12.
   3. The nucleic acid construct of embodiment 1, wherein the SARS-CoV-2 antigen comprises a sequence selected from the group consisting of SEQ ID NO: 4, SEQ ID NO: 5, and SEQ ID NO: 6.
   4. A nucleic acid construct for heterologous expression of a SARS CoV-2 antigen on the surface of a yeast cell, the nucleic acid construct comprising:
(a) a promoter element active in a yeast cell; and
(b) one or more heterologous polynucleotides encoding (i) a SARS-CoV-2 antigen; and (ii) a yeast surface display polypeptide or fragment thereof,

wherein the one or more heterologous polynucleotides are operably linked to the promoter element, and

wherein the one or more heterologous polynucleotides encodes a polypeptide comprising a sequence as set forth in SEQ ID NO: 13.

5. The nucleic acid sequence of embodiment 4, wherein the one or more heterologous polynucleotides encodes a polypeptide consisting of a sequence as set forth in SEQ ID NO: 13.
6. The nucleic acid construct of embodiment 5, wherein the one or more heterologous polynucleotides comprises a sequence at least 99% identical to SEQ ID NO: 20.
7. A nucleic acid construct for heterologous expression of a SARS CoV-2 antigen on the surface of a yeast cell, the nucleic acid construct comprising:

(a) a promoter element active in a yeast cell; and

(b) one or more heterologous polynucleotides encoding (i) a SARS-CoV-2 antigen; and (ii) a yeast surface display polypeptide or fragment thereof,

wherein the one or more heterologous polynucleotides are operably linked to the promoter element, and

wherein the one or more heterologous polynucleotides encodes a polypeptide comprising a sequence as set forth in SEQ ID NO: 14.

8. The nucleic acid sequence of embodiment 7, wherein the one or more heterologous polynucleotides encodes a polypeptide consisting of a sequence as set forth in SEQ ID NO: 14.
9. The nucleic acid construct of embodiment 8, wherein the one or more heterologous polynucleotides comprises a sequence at least 99% identical to SEQ ID NO: 21.
10. A nucleic acid construct for heterologous expression of a SARS CoV-2 antigen on the surface of a yeast cell, the nucleic acid construct comprising:

(a) a promoter element active in a yeast cell; and

(b) one or more heterologous polynucleotides encoding (i) a SARS-CoV-2 antigen; and (ii) a yeast surface display polypeptide or fragment thereof,

wherein the one or more heterologous polynucleotides are operably linked to the promoter element, and

wherein the one or more heterologous polynucleotides encodes a polypeptide comprising a sequence as set forth in SEQ ID NO: 15.

11. The nucleic acid sequence of embodiment 10, wherein the one or more heterologous polynucleotides encodes a polypeptide consisting of a sequence as set forth in SEQ ID NO: 15.
12. The nucleic acid construct of embodiment 11, wherein the one or more heterologous polynucleotides comprises a sequence at least 99% identical to SEQ ID NO: 22.
13. A nucleic acid construct for heterologous expression of a SARS CoV-2 antigen on the surface of a yeast cell, the nucleic acid construct comprising:
(a) a promoter element active in a yeast cell; and
(b) one or more heterologous polynucleotides encoding (i) one or more SARS-CoV-2 antigens and (ii) a yeast surface display polypeptide or fragment thereof,
   wherein the one or more heterologous polynucleotides are operably linked to the promoter element.
   14. The nucleic acid construct of embodiment 13, wherein the one or more SARS-CoV-2 antigens are from the SARS-CoV-2 S protein.
   15. The nucleic acid construct of embodiment 14, wherein the polynucleotide encodes a polypeptide having at least 75% sequence identity to the amino acid sequence set forth in SEQ ID NO: 4.
   16. The nucleic acid construct of embodiment 15, wherein the polynucleotide encodes a polypeptide having at least 80% sequence identity to the amino acid sequence set forth in SEQ ID NO: 4.
   17. The nucleic acid construct of embodiment 16, wherein the polynucleotide encodes a polypeptide having at least 85% sequence identity to the amino acid sequence set forth in SEQ ID NO: 4.
   18. The nucleic acid construct of embodiment 17, wherein the polynucleotide encodes a polypeptide having at least 90% sequence identity to the amino acid sequence set forth in SEQ ID NO: 4.
   19. The nucleic acid construct of embodiment 18, wherein the polynucleotide encodes a polypeptide having at least 95% sequence identity to the amino acid sequence set forth in SEQ ID NO: 4.
   20. The nucleic acid construct of embodiment 19, wherein the polynucleotide encodes a polypeptide having at least 98% sequence identity to the amino acid sequence set forth in SEQ ID NO: 4.
   21. The nucleic acid construct of embodiment 20, wherein the polypeptide comprises a full-length SARS-CoV-2 S protein having the amino acid sequence set forth in SEQ ID NO: 4.
   22. The nucleic acid construct of embodiment 14, wherein the one or more SARS-CoV-2 antigens are from the SARS-CoV-2 S1 subunit.
   23. The nucleic acid construct of embodiment 22, wherein the polynucleotide encodes a polypeptide having at least 75% sequence identity to the amino acid sequence set forth in SEQ ID NO: 5.
   24. The nucleic acid construct of embodiment 23, wherein the polynucleotide encodes a polypeptide having at least 80% sequence identity to the amino acid sequence set forth in SEQ ID NO: 5.
   25. The nucleic acid construct of embodiment 24, wherein the polynucleotide encodes a polypeptide having at least 85% sequence identity to the amino acid sequence set forth in SEQ ID NO: 5.
   26. The nucleic acid construct of embodiment 25, wherein the polynucleotide encodes a polypeptide having at least 90% sequence identity to the amino acid sequence set forth in SEQ ID NO: 5.
   27. The nucleic acid construct of embodiment 26, wherein the polynucleotide encodes a polypeptide having at least 95% sequence identity to the amino acid sequence set forth in SEQ ID NO: 5.
   28. The nucleic acid construct of embodiment 27, wherein the polynucleotide encodes a polypeptide having at least 98% sequence identity to the amino acid sequence set forth in SEQ ID NO: 5.
   29. The nucleic acid construct of embodiment 28, wherein the polypeptide comprises a full-length SARS-CoV-2 S1 subunit having the amino acid sequence set forth in SEQ ID NO: 5.
   30. The nucleic acid construct of embodiment 22, wherein the one or more SARS-CoV-2 antigens are from the SARS-CoV-2 RBD.
   31. The nucleic acid construct of embodiment 30, wherein the polynucleotide encodes a polypeptide having at least 75% sequence identity to the amino acid sequence set forth in SEQ ID NO: 6.
   32. The nucleic acid construct of embodiment 31, wherein the polynucleotide encodes a polypeptide having at least 80% sequence identity to the amino acid sequence set forth in SEQ ID NO: 6.
   33. The nucleic acid construct of embodiment 32, wherein the polynucleotide encodes a polypeptide having at least 85% sequence identity to the amino acid sequence set forth in SEQ ID NO: 6.
   34. The nucleic acid construct of embodiment 33, wherein the polynucleotide encodes a polypeptide having at least 90% sequence identity to the amino acid sequence set forth in SEQ ID NO: 6.
   35. The nucleic acid construct of embodiment 34, wherein the polynucleotide encodes a polypeptide having at least 95% sequence identity to the amino acid sequence set forth in SEQ ID NO: 6.
   36. The nucleic acid construct of embodiment 35, wherein the polynucleotide encodes a polypeptide having at least 98% sequence identity to the amino acid sequence set forth in SEQ ID NO: 6.
   37. The nucleic acid construct of embodiment 36, wherein the polypeptide comprises a full-length SARS-CoV-2 RBD having the amino acid sequence set forth in SEQ ID NO: 6.
   38. The nucleic acid construct of any one of embodiments 13-37, wherein the nucleotide sequence of one or more heterologous polynucleotides is at least 70% identical to a sequence as set forth in SEQ ID NO: 1, SEQ ID NO: 2, or SEQ ID NO: 3.
   39. The nucleic acid construct of embodiment 38, wherein the nucleotide sequence of one or more heterologous polynucleotides is at least 80% identical to a sequence as set forth in SEQ ID NO: 1, SEQ ID NO: 2, or SEQ ID NO: 3.
   40. The nucleic acid construct of embodiment 39, wherein the nucleotide sequence of one or more heterologous polynucleotides is at least 90% identical to a sequence as set forth in SEQ ID NO: 1, SEQ ID NO: 2, or SEQ ID NO: 3.
   41. The nucleic acid construct of embodiment 40, wherein the nucleotide sequence of one or more heterologous polynucleotides is at least 95% identical to a sequence as set forth in SEQ ID NO: 1, SEQ ID NO: 2, or SEQ ID NO: 3.
   42. The nucleic acid construct of embodiment 41, wherein the nucleotide sequence of one or more heterologous polynucleotides comprises a sequence set forth in SEQ ID NO: 1, SEQ ID NO: 2, or SEQ ID NO: 3.
   43. The nucleic acid construct of any one of embodiments 13-42, wherein the yeast surface display polypeptide is an a-agglutinin polypeptide or fragment thereof.
   44. The nucleic acid construct of embodiment 43, wherein the a-agglutinin polypeptide or fragment thereof is an Aga2 peptide.
   45. The nucleic acid construct of embodiment 44, wherein the Aga2 peptide has the amino acid sequence set forth in SEQ ID NO: 7.
   46. The nucleic acid construct of any one of embodiments 25-57, wherein the one or more heterologous polynucleotides further encode a signal peptide in-frame with the SARS-CoV-2 antigen.
   47. The nucleic acid construct of embodiment 46, wherein the signal peptide is an Aga2 signal peptide.
   48. The nucleic acid construct of any one of embodiments 13-47, further comprising a coding sequence for a linker peptide.
   49. The nucleic acid construct of embodiment 48, wherein the linker peptide sequence comprises (GGGGS)_{N}, wherein N is between 1 and 10, inclusive.
   50. The nucleic acid construct of embodiment 49, wherein N is 3.
   51. The nucleic acid construct of any one of embodiments 13-50, wherein the one or more heterologous polynucleotides further encode one or more tags.
   52. The nucleic acid construct of embodiment 51, wherein the one or more tags comprises an epitope tag.
   53. The nucleic acid construct of embodiment 52, wherein the epitope tag is a V5 epitope tag.
   54. The nucleotide acid construct of embodiment 53, wherein the V5 epitope tag has the amino acid sequence set forth in SEQ ID NO: 9.
   55. The nucleic acid construct of any one of embodiments 51-54, wherein the one or more tags comprises 6×His tag.
   56. The nucleic acid construct of any one of embodiments 13-55, wherein the one or more heterologous polynucleotides encode a polypeptide having the yeast surface display polypeptide at a first terminus.
   57. The nucleic acid construct of embodiment 56, wherein the polypeptide has, at a second terminus, (i) a tag, (ii) signal peptide, or (iii) the SARS-CoV-2 antigen.
   58. The nucleic acid construct of embodiment 57,

wherein the polypeptide has, at the second terminus, a signal peptide, and

wherein cleavage of the signal peptide results in a mature polypeptide having the SARS-CoV-2 antigen at the second terminus.

59. The nucleic acid construct of any one of embodiments 56-58, wherein the first terminus is the C-terminus.
60. The nucleic acid construct of any one of embodiments 56-58, wherein the first terminus is the N-terminus.
61. The nucleic acid construct of embodiment 43, wherein the one or more heterologous polynucleotides encodes a polypeptide comprising, in the N→C direction:

(a) an Aga2 signal peptide;

(b) the one or more SARS-CoV-2 antigens;

(c) a linker peptide sequence; and

(d) an Aga2 peptide;

wherein cleavage of the Aga2 signal peptide results in a mature polypeptide having the SARS-CoV-2 antigen at the N-terminus.

62. The nucleic acid construct of embodiment 61, wherein the linker peptide sequence comprises (GGGGS)_{N}, wherein N is between 1 and 10.
63. The nucleic acid construct of embodiment 62, wherein N is 3.
64. The method of any one of embodiments 61-63, wherein the Aga2 peptide has the amino acid sequence set forth in SEQ ID NO: 7.
65. The nucleic acid construct of embodiment 43, wherein the one or more heterologous polynucleotides encodes a polypeptide comprising, in the N→C direction:

(a) an Aga2 signal peptide,

(b) an Aga2 peptide,

(c) a linker peptide, and

(d) the one or more SARS-CoV-2 antigens,
wherein cleavage of the Aga2 signal peptide results in a mature polypeptide having the Aga2 peptide at the N-terminus.
   66. The nucleic acid construct of embodiment 65, wherein the polypeptide further comprises one or more tags located C-terminal to the one or more SARS-CoV-2 antigens.
   67. The nucleic acid construct of embodiment 66, wherein the one or more tags comprise a V5 epitope tag.
   68. The nucleic acid construct of embodiment 66 or 67, wherein the one or more tags comprise a 6×His epitope tag.
   69. The nucleic acid construct of any one of embodiments 65-68, wherein the linker peptide comprises the sequence (GGGGS)_{N}, wherein N is between 1 and 10.
   70. The nucleic acid construct of embodiment 69, wherein N is 3.
   71. The nucleic acid of any one of embodiments 65-70, wherein the Aga2 peptide has the amino acid sequence set forth in SEQ ID NO: 7.
   72. A polypeptide expressed by the nucleic acid construct of any one of embodiments 1-63, or a mature version thereof.
   73. The polypeptide of embodiment 72, wherein the polypeptide comprises, in the N→C direction, an Aga2 signal peptide, a SARS-CoV-2 antigen, a V5 epitope, a (G₄S)_{N} linker, and an Aga2p subunit, wherein N is between 1 and 10.
   74. The polypeptide of embodiment 72, wherein the polypeptide comprises, in the N→C direction, a SARS-CoV-2 antigen, a V5 epitope, a (G₄S)_{N} linker, and an Aga2p subunit, wherein N is between 1 and 10.
   75. The polypeptide of embodiment 73 or 74, wherein N is 3.
   76. A recombinant yeast comprising the nucleic acid construct of any one of embodiments 1-63 or the polypeptide of any one of embodiments 72-75.
   77. The recombinant yeast of embodiment 76, wherein the recombinant yeast further comprises a nucleic acid encoding a second yeast surface display polypeptide.
   78. The recombinant yeast of embodiment 77, wherein the second yeast surface display polypeptide is operably linked to an inducible promoter.
   79. The recombinant yeast of embodiment 77 or 78, wherein the second yeast surface display polypeptide is an a-agglutinin polypeptide or fragment thereof.
   80. The recombinant yeast of embodiment 79, wherein the a-agglutinin polypeptide or fragment thereof is an Aga1 peptide.
   81. The recombinant yeast of any one of embodiments 76-80, wherein the yeast is S. cerevisiae.
   82. The recombinant yeast of embodiments 81, wherein the S. cerevisiae is strain EBY100.
   83. The recombinant yeast of any one of embodiments 76-82, wherein the recombinant yeast displays the one or more SARS-CoV-2 antigens on its cell surface.
   84. The recombinant yeast of embodiment 83, wherein the one or more SARS-CoV-2 antigens is from the SARS-CoV-2 S protein.
   85. The recombinant yeast of embodiment 84, wherein the one or more SARS-CoV-2 antigens is from the SARS-CoV-2 S1 subunit.
   86. The recombinant yeast of embodiment 85, wherein the one or more SARS-CoV-2 antigens is from the SARS-CoV-2 RBD.
   87. A vaccine composition comprising an effective amount of the recombinant yeast of any one of embodiment 76-86, or an extract thereof.
   88. The vaccine composition of embodiment 87, further comprising an adjuvant.
   89. The vaccine composition of embodiment 87, wherein the composition lacks an adjuvant.
   90. An oral dosage formulation comprising the vaccine composition of embodiment 87, 88, or 89.
   91. The oral dosage formulation of embodiment 90, further comprising an oral solid dosage form excipient.
   92. The oral dosage formulation of embodiment 91, wherein the oral solid dosage form excipient is selected from the group consisting of binders and fillers, coatings, lubricants, matrix formers, and disintegrants.
   93. The oral dosage formulation of embodiment 90, wherein the formulation is a liquid or gel formulation.
   94. The oral dosage formulation of embodiment 93, wherein the liquid or gel formulation is in a monophasic form.
   95. The oral dosage formulation of embodiment 94, wherein the monophasic form is selected from the group consisting of aqueous solutions and non-aqueous solutions.
   96. The oral dosage formulation of embodiment 93, wherein the formulation is in a biphasic form.
   97. The oral dosage formulation of embodiment 96, wherein the biphasic form is selected from the group consisting of suspensions, emulsions, and mixtures.
   98. A method of inducing an antigen-specific immune response to SARS-CoV-2 in a subject, comprising administering to the subject an effective amount of a vaccine composition or oral dosage formulation of any one of embodiments 87-97.
   99. The method of embodiment 98, wherein the subject has not tested positive for SARS-CoV-2.
   100. The method of embodiment 98 or 99, wherein the subject is a human subject.
   101. The method of any one of embodiments 98-100, wherein the administering comprises oral administration.

What is claimed is:

1. A nucleic acid construct for heterologous expression of a SARS CoV-2 antigen on the surface of a yeast cell, the nucleic acid construct comprising:
(a) a promoter element active in a yeast cell; and
(b) one or more heterologous polynucleotides encoding (i) one or more SARS-CoV-2 antigens and (ii) a yeast surface display polypeptide or fragment thereof,
wherein the one or more heterologous polynucleotides are operably linked to the promoter element.

2. The nucleic acid construct of claim 1, wherein the one or more SARS-CoV-2 antigens are from the SARS-CoV-2 S protein.

3. The nucleic acid construct of claim 2, wherein the polynucleotide encodes a polypeptide having at least 75% sequence identity to the amino acid sequence set forth in SEQ ID NO: 4.

4. The nucleic acid construct of claim 2, wherein the one or more SARS-CoV-2 antigens are from the SARS-CoV-2 S1 subunit.

5. The nucleic acid construct of claim 4, wherein the polynucleotide encodes a polypeptide having at least 75% sequence identity to the amino acid sequence set forth in SEQ ID NO: 5.

6. The nucleic acid construct of claim 4, wherein the one or more SARS-CoV-2 antigens are from the SARS-CoV-2 RBD.

7. The nucleic acid construct of claim 6, wherein the polynucleotide encodes a polypeptide having at least 75% sequence identity to the amino acid sequence set forth in SEQ ID NO: 6.

8. The nucleic acid construct of claim 1, wherein the yeast surface display polypeptide is an a-agglutinin polypeptide or fragment thereof.

9. The nucleic acid construct of claim 8, wherein the a-agglutinin polypeptide or fragment thereof is an Aga2 peptide.

10. The nucleic acid construct of any one of claim 1,
wherein the one or more heterologous polynucleotides encode a polypeptide having the yeast surface display polypeptide at a first terminus and a signal peptide at a second terminus, and
wherein cleavage of the signal peptide results in a mature polypeptide having the SARS-CoV-2 antigen at the second terminus.

11. The nucleic acid construct of claim 10, wherein the one or more heterologous polynucleotides encodes a polypeptide comprising, in the N→C direction:
(a) an Aga2 signal peptide;
(b) the one or more SARS-CoV-2 antigens;
(c) a linker peptide sequence; and
(d) an Aga2 peptide;
wherein cleavage of the Aga2 signal peptide results in a mature polypeptide having the SARS-CoV-2 antigen at the N-terminus.

12. The nucleic acid construct of claim 1, wherein the one or more heterologous polynucleotides encodes a polypeptide comprising, in the N→C direction:
(a) an Aga2 signal peptide,
(b) an Aga2 peptide,
(c) a linker peptide, and
(d) the one or more SARS-CoV-2 antigens,
wherein cleavage of the Aga2 signal peptide results in a mature polypeptide having the Aga2 peptide at the N-terminus.

13. A nucleic acid construct for heterologous expression of a SARS CoV-2 antigen on the surface of a yeast cell, the nucleic acid construct comprising:
(a) a promoter element active in a yeast cell; and
(b) one or more heterologous polynucleotides encoding (i) a SARS-CoV-2 antigen; and (ii) a yeast surface display polypeptide or fragment thereof,
wherein the one or more heterologous polynucleotides are operably linked to the promoter element, and
wherein the one or more heterologous polynucleotides encodes a polypeptide comprising, in the N→C direction:
(1) an Aga2 signal peptide having the amino acid sequence of SEQ ID NO: 10;
(2) the SARS-CoV-2 antigen; and
(3) a polypeptide having the amino acid sequence of SEQ ID NO: 12.

14. The nucleic acid construct of claim 13, wherein the one or more heterologous polynucleotides encodes a polypeptide consisting of, in the N→C direction:
(1) an Aga2 signal peptide having the amino acid sequence of SEQ ID NO: 10;
(2) the SARS-CoV-2 antigen; and
(3) a polypeptide having the amino acid sequence of SEQ ID NO: 12.

15. The nucleic acid construct of claim 13, wherein the SARS-CoV-2 antigen comprises a sequence selected from the group consisting of SEQ ID NO: 4, SEQ ID NO: 5, and SEQ ID NO: 6.

16. The nucleic acid construct of claim 15, wherein the one or more heterologous polynucleotides encodes a polypeptide comprising a sequence as set forth in SEQ ID NO: 13.

17. The nucleic acid construct of claim 16, wherein the one or more heterologous polynucleotides comprises a sequence at least 99% identical to SEQ ID NO: 20.

18. The nucleic acid construct of claim 15, wherein the one or more heterologous polynucleotides encodes a polypeptide comprising a sequence as set forth in SEQ ID NO: 14.

19. The nucleic acid construct of claim 18, wherein the one or more heterologous polynucleotides comprises a sequence at least 99% identical to SEQ ID NO: 21.

20. The nucleic acid construct of claim 15, wherein the one or more heterologous polynucleotides encodes a polypeptide comprising a sequence as set forth in SEQ ID NO: 15.

21. The nucleic acid construct of claim 20, wherein the one or more heterologous polynucleotides comprises a sequence at least 99% identical to SEQ ID NO: 22.

22. A polypeptide expressed by the nucleic acid construct of claim 1, or a mature version thereof.

23. A recombinant yeast comprising the nucleic acid construct of claim 1.

24. The recombinant yeast of claim 23, wherein the recombinant yeast displays the one or more SARS-CoV-2 antigens on its cell surface.

25. The recombinant yeast of claim 24, wherein the one or more SARS-CoV-2 antigens is from the SARS-CoV-2 S protein.

26. The recombinant yeast of claim 25, wherein the one or more SARS-CoV-2 antigens is from the SARS-CoV-2 S1 subunit.

27. The recombinant yeast of claim 26, wherein the one or more SARS-CoV-2 antigens is from the SARS-CoV-2 RBD.

28. A vaccine composition comprising an effective amount of the recombinant yeast of claim 23, or an extract thereof.

29. An oral dosage formulation comprising the vaccine composition of claim 28.

30. A method of inducing an antigen-specific immune response to SARS-CoV-2 in a subject, comprising administering to the subject an effective amount of a vaccine composition of claim 28.
